(19) United States
(12) Patent Application Publication
(10) Pub. No.: US 2026/0256705 A1
(43) Pub. Date: Sep. 3, 2026

(54) LIPID NANOPARTICLE FORMULATIONS FOR VACCINES

(71) Applicant: GLOBAL LIFE SCIENCES SOLUTIONS CANADA ULC, Vancouver (CA)

(72) Inventors: Pierrot Harvie, Vancouver (CA); Lloyd Brian Jeffs, Vancouver (CA); Ruo Yu Zhang, Vancouver (CA); Mohammadreza Kazemian, Vancouver (CA); Harish Chakrapani, Vancouver (CA); Nikita Jain, Vancouver (CA); Anitha Thomas, Vancouver (CA)

(21) Appl. No.: 18/875,296

(22) PCT Filed: Jun. 28, 2023
(86) PCT No.: PCT/US2023/069303

§ 371 Date: Dec. 16, 2024

Related U.S. Application Data
(60) Provisional application No. 63/357,094, filed on Jun. 30, 2022.

(51) Int. Cl. A61K 9/51; A61K 9/19; A61K 39/00; A61K 39/145; A61K 39/215; A61K 47/10; A61K 47/24; A61K 47/28

(52) U.S. Cl. CPC A61K 9/5123; A61K 9/19; A61K 39/145; A61K 39/215; A61K 47/10; A61K 47/24; A61K 47/28; A61K 2039/53

(57) ABSTRACT

Provided is a lipid formulation capable of forming a lipid-based nanoparticle comprising an ionizable lipid to phospholipid molar ratio of 0.1-1.30 of in association with a nucleic acid payload, and in some embodiments, a stabilizing agent. In embodiments, the nucleic acid payload is a vaccine genetic element.

### CROSS-REFERENCE TO RELATED APPLICATIONS

This patent application claims the benefit of U.S. Provisional Patent Application No. 63/357,094, filed Jun. 30, 2022, which is incorporated by reference.

### BACKGROUND

### Field of the Invention

The field of the invention relates methods and lipid formulations suitable for forming RNA-based vaccines in lipid nanoparticles.

### Related Art

Nucleic-acid-based vaccines offer advantages over traditional vaccines in terms of safety and efficacy. RNA vaccines are subject to degradation by exonucleases and endonucleases in vivo without a delivery system, so they need a carrier.

Currently, lipid nanoparticles (LNP) are among the most frequently used vectors for in vivo RNA delivery. Lipid nanoparticles or LNP generally consist of different lipids, each serving distinct functions. These LNP can have a lipidic or aqueous core and may contain bilayer structures depending on the abundance of each type of lipids.

The components of the LNP formulations are generally: ionizable cationic lipids, which spontaneously encapsulate negatively-charged mRNA via by a combination of attractive electrostatic interactions with RNA and hydrophobic interactions; neutral phospholipids to reduce charge-related toxicity and to maintain structure of the LNP; and cholesterol to stabilize the LNP and help with cell entry, and a lipid-conjugated polyethylene glycol (PEG).

The properties of individual LNP vary. Diffusive or bulk mixing can lead to LNP with variable compositions. Therefore, rapid mixing of the ethanol-lipid phase with mRNA in excess water is key for the synthesis of small, uniform LNP. The Precision Nanoystems ULC NanoAssemblr® line of mixers is recommended for this purpose.

Recognized challenges to creating successful LNP vaccines are safety, manufacturability, stability, and efficacy. Current mRNA vaccines must be kept frozen at low temperatures for storage. Some cases of adverse events have been reported for LNP vaccines, which has resulted in resistance to vaccination by some.

A better LNP form of vaccine with increased stability is still required.

### SUMMARY OF THE INVENTION

In accordance with one embodiment of the invention, there is provided a lipid formulation capable of forming a lipid-based nanoparticle suitable for vaccines, the lipid formulation comprising an ionizable lipid to phospholipid ratio of 0.10-1.30 mol:mol, in some embodiments, a ratio of 0.33-1.20 mol:mol, in some embodiments, a ratio of 0.10-0.70 mol:mol, and in some embodiments, a ratio of 0.40-0.70 mol:mol. In embodiments, the lipid formulation further includes a nucleic acid payload. In embodiments, the nucleic acid payload is a nucleic acid vaccine element. In some embodiments, the lipid formulation further comprises a stabilizer. In some embodiments, the lipid formulation further comprises a sterol.

In embodiments, the molar ratio of ionizable lipid to phospholipid is about 1.30, or about 1.29, or about 1.28, or about 1.27, or about 1.26, or about 1.25, or about 1.24, or about 1.23, or about 1.22, or about 1.21, or about 1.20, or about 1.19, or about 1.19, or about 1.18, or about 1.17, or about 1.16, or about 1.15, or about 1.14, or about 1.13, or about 1.12, or about 1.11, or about 1.10, or about 1.09, or about 1.08, or about 1.07, or about 1.06, or about 1.05, or about 1.04, or about 1.03, or about 1.02, or about 1.01, or about 1.00, or about 0.99, or about 0.98, or about 0.97, or about 0.96, or about 0.95, or about 0.94, or about 0.93, or about 0.92, or about 0.91, or about 0.90, or about 0.89, or about 0.88, or about 0.87, or about 0.86, or about 0.85, or about 0.84, or about 0.83, or about 0.82, or about 0.81, or about 0.80, or about 0.79, or about 0.78, or about 0.77, or about 0.76, or about 0.75, or about 0.74, or about 0.73, or about 0.72, or about 0.71.

In embodiments, the molar ratio or ionizable lipid to phospholipid is about 0.70, or about 0.69, or about 0.68, or about 0.67, or about 0.66, or about 0.65, or about 0.64, or about 0.63, or about 0.62, or about 0.61, or about 0.60, or about 0.59, or about 0.58, or about 0.57, or about 0.56, or about 0.55, or about 0.54, or about 0.53, or about 0.52.

In embodiments, the ionizable lipid to phospholipid ratio is about 0.51, or about 0.50, or about 0.49, or about 0.48, or about 0.47, or about 0.46, or about 0.45, or about 0.44, or about 0.43, or about 0.42, or about 0.41, or about 0.40.

In embodiments, the ionizable lipid to phospholipid ratio is about 0.39, or about 0.38, or about 0.37, or about 0.36, or about 0.35, or about 0.34, or about 0.33, or about 0.32, or about 0.31, or about 0.30.

In embodiments, the ionizable lipid to phospholipid ratio is about 0.29, or about 0.28, or about 0.27, or about 0.26, or about 0.25, or about 0.24, or about 0.23, or about 0.22, or about 0.21, or about 0.20.

In embodiments, the ionizable lipid to phospholipid ratio is about 0.19, or about 0.18, or about 0.17, or about 0.16, or about 0.15, or about 0.14, or about 0.13, or about 0.12, or about 0.11, or about 0.10.

In accordance with one embodiment of the invention, there is provided a lipid formulation, wherein an ionizable lipid makes up about 20-40 mol % of the total composition.

In accordance with one embodiment of the invention, there is provided a lipid formulation, wherein the ionizable lipid comprises a mixture of ionizable lipids. In embodiments, the phospholipid makes up about 25-60 mol % of the total formulation.

In accordance with one embodiment of the invention, sterol such as cholesterol or cholesteryl hemisuccinate comprises from 15-25 mol % of the total composition.

In accordance with one embodiment of the invention, there is provided a lipid formulation, wherein the stabilizer comprises from 0.0-2.5 mole % of the total volume of the lipid formulation.

According to the invention, there is provided a lipid formulation for encapsulating a nucleic acid payload in a nanoparticle, the formulation including an ionizable lipid, a sterol, and a phospholipid, wherein the phospholipid content is between 25-60% of the nanoparticle, and the ionizable to phospholipid molar ratio is from 0.33 to 1.2. In embodiments, the nanoparticle is a vaccine.

In embodiments, the stabilizer is a PEG lipid, in some embodiments PEG-DMPG. In embodiments, the phospholipid is DSPC. In embodiments, the ionizable lipid is selected from the group consisting of PNI 516, PNI 550, PNI 127, PNI 560, PNI 580, PNI 659, PNI 721, PNI 722, PNI 726, PNI 728, and PNI 730. In embodiments, the sterol is cholesterol.

According to the invention, there is provided a vaccine including a lipid formulation for encapsulating a nucleic acid payload, the vaccine including an ionizable lipid, a sterol, and a phospholipid, and wherein the phospholipid content is between 25-60% of the vaccine, and the ionizable to phospholipid molar ratio is from 0.33 to 1.2. In embodiments, the nucleic acid payload codes for an antigen selected from coronavirus spike protein and influenza hemagglutinin protein.

In embodiments, the nucleic acid payload is derived from influenza virus mRNA. In embodiments, the nucleic acid payload is derived from coronavirus.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 is a graphical representation of SARsCOV-2 protein expression dose response following Vaccmixb PNI516 and V46-PNI 516 transfection in BHK 570 cells. The dose response curve was created by with RNA doses of from 1000 to 0.49 ng/ml;

FIG. 2 is a graphical representation of SARsCOV-2 protein expression dose response following LNP transfection in BHK 570 cells with various PNI516 LNP formulations containing nCoV PNI A5 saRNA from 1000 to 0.49 ng/ml;

FIG. 3 is a scatter plot graph of an ELISA of SARsCOV-2 spike protein specific IgG expression in BALB/c mouse serum 21 days post intramuscular LNP injection at 1 ug/mouse; and

FIG. 4 is a reproduction of Cryo-TEM imaging for A) Vaccmixb PNI 516, B) V46-PNI 516, and C) V47-PNI 516 LNP.

FIG. 5 is a graphical representation of in vitro transfection ability of saRNA LNP with Vaccmixb, V46-PNI 516, V47-PNI 516, and V22-PNI 516 following addition of Jurkat cells to GFP saRA LNPs.

FIG. 6 is a graphical representation of in vitro transfection ability of mRNA LNP with V47-PNI 516, V46-PNI 516, V02-PNI 516 and V22-PNI 516 following addition of Jurkat cells to GFP mRNA LNP.

FIG. 7 is a graphical representation of transfection ability of EGFP saRNA LNP with expansion medium supplemented with activators/recombinant human IL-2.

FIG. 8 is photographs of mice that have had LNP with a luciferase mRNA payload injected into them intravenously at 0.1 mg/kg. The top two photographs show fluorescence at four hours post administration of LNP comprising PNI 516 30%, DPSC 56%, cholesterol 12.5%, and DMP-PEG 1.5%. The bottom two photographs, which are brighter than the top two paragraphs, show fluorescence at four house post administration of LNP comprising PNI 580 30%, DSPC 56%, cholesterol 12.5%, and DMG-PEG 1.5%.

### DETAILED DESCRIPTION OF THE INVENTION

In accordance with an embodiment of the invention, there are provided lipid mix formulations for use in generating more effective lipid-based formulations of nucleic acid cargo (including vaccines) and other oligomers such as peptides, and methods for using these lipid mixes and resulting formulations to prepare vaccines. The lipid mixes have an unusual ratio of ionizable lipid to phospholipid as compared to established optimal ratios of 0.2, yet surprisingly measure as being more effective.

TABLE 1
Prior Art Exemplary Lipid Mixes

| Lipid | Ionizable | Phospho- | | | |
|---|---|---|---|---|---|
| Component | Lipid, | lipid, | Sterol | Stabilizer | IL/DSPC |
| Mixture | Mol % | Mol % | Mol % | Mol % | Ratio |
| Onpattro ® | MC3, 50 | DSPC, | Choles- | DMG- | 5 |
| | | 10 | terol, | PEG2000 | |
| | | | 38.5 | 1.5 | |
| Spikevax ™ | SM-102, | DSPC, | Choles- | DMG- | 5 |
| | 50 | 10 | terol, | PEG2000 | |
| | | | 38.5 | 1.5 | |
| Corminaty ™ | ALC-315 | DSPC, | Choles- | ALC-519 | 4.9 |
| | 46.3 | 9.4 | terol, | 1.6 | |
| | | | 42.7 | | |

In another aspect, the lipid mix formulations of the invention are provided for mixing with nucleic acid vaccine elements to create a lipid nucleic acid particle which enhances delivery of the nucleic acid into target cells or tissues, with less toxicity and greater ease of manufacture than lipid nucleic acid particles such as those made from commercially available lipid mixes such as Lipofectamine™ transfecting agent.

In another aspect, the invention provides lipid mix formulations including ionizable lipid, one or more phospholipid(s), cholesterol, and optionally a stabilizing agent.

In another aspect, the lipid mix formulations according the invention are provided for formulating vaccines.

In another aspect, the invention provides lipid mix formulations for formulating mRNA LNPs.

Self-amplifying mRNA (saRNA) has the advantage of prolonged translation and high yield of target antigen compared to regular mRNA vaccines like Moderna mRNA-1273 (Spikevax) and Pfizer-Biontech's BNT162b2 (Comirnaty) (see Table 1). One example of a nucleic acid vaccine element is self-amplifying mRNA as described in PCT Pub. No. WO23057979 by Abraham et al.

A review of the “severe acute respiratory syndrome coronavirus 2” (SARS-CoV-2) genetics includes spike protein genetics. The spike protein has proven a useful target for SARS-CoV-2 vaccines, and is of interest for variants of the virus that continue to cause illness. A modified spike protein gene sequence was used herein.

In this disclosure, the word “comprising” is used in a non-limiting sense to mean that items following the word are included, but items not specifically mentioned are not excluded. It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

In this disclosure, the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.). In this disclosure the singular forms an “an”, and “the” include plural referents unless the content clearly dictates otherwise. Thus, for example, reference to a composition containing “a compound” includes a mixture of two or more compounds.

In this disclosure, term “or” is generally employed in its sense including “and/or” unless the content clearly dictates otherwise.

As used herein, the term “about” is defined as meaning 12.5% plus or minus the recited number. It is used to signify that the desired target concentration might be, for example, 40 Mol %, but that through mixing inconsistencies, the actual percentage might differ by +/−5 Mol %.

As used herein, the term “substantially” is defined as being 5% plus or minus the recited number. It is used to signify that the desired target concentration might be, for example, 40 Mol %, but that through mixing inconsistencies, the actual percentage might differ by +/−2 Mol %.

As used herein, the term “nucleic acid cargo” is defined as a substance intended to have a direct effect in the mitigation or prevention of disease, or to act as a research reagent. In preferred embodiments, the nucleic acid cargo is an mRNA, or saRNA In preferred embodiments, the therapeutic agent is a nucleic acid therapeutic, such as an RNA polynucleotide. In preferred embodiments, the therapeutic agent is messenger RNA (mRNA) or self-amplifying RNA (saRNA). In preferred embodiments, the therapeutic agent is double stranded circular DNA (plasmid), linearized plasmid DNA, minicircles or msDNA (multicopy single stranded DNA).

In this disclosure, the word “comprising” is used in a non-limiting sense to mean that items following the word are included, but items not specifically mentioned are not excluded. It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

In this disclosure, “transfection” means the transfer of nucleic acid into cells for the purpose of inducing the expression of a specific gene(s) of interest in both laboratory and clinical settings. It typically includes an ionizable lipid to associate with nucleic acid, and phospholipids. LIPOFECTIN™ and LIPOFECTAMINE™ are established commercial transfecting reagents sold by ThermoFisher Scientific. These research reagents contain permanently cationic lipid/s and are not suitable for use in or ex vivo.

In this disclosure the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.). In this disclosure the singular forms an “an”, and “the” include plural referents unless the content clearly dictates otherwise. Thus, for example, reference to a composition containing “a compound” includes a mixture of two or more compounds. In this disclosure term “or” is generally employed in its sense including “and/or” unless the content clearly dictates otherwise.

“nCoV PNI A5 saRNA” is a SARS Cov2 spike protein expressing saRNA incorporated into a Venezuelan Equine Encephalitis Virus TC83 Replicon with subgenomic promoter containing a multiple cloning site to insert any GOIs as described in PCT Pub. No. WO23057979 by Abraham et al.

“Lipid” refers to a structurally diverse group of organic compounds that are fatty acid derivatives or sterols or could be lipid like materials as in lipidoids (example C12-200) and are characterized by being insoluble in water but soluble in many organic solvents.

“Lipid mix formulations”. Lipid mix formulations refers to the types of components, ratios of components, and the ratio of the total components to the nucleic acid payloads. For example, a lipid mix formulation of 30 Mol % ionizable lipid, 50 Mol % phospholipid, 20 Mol % sterol, and 1.5 Mol % stabilizing agent would be a lipid mix formulation. In preferred embodiments, the lipid mix formulation is 28.7 mol % IL/49.8 mol % DSPC/20 mol % Cholesterol/1.5 mol % PEG-DMG.

“Lipid Particles” or “Lipid Nanoparticles” or “LNP”. The invention provides lipid particles manufactured from the lipid mix formulations described above and illustrated below. The lipid particle represents the physical organization of the lipid mix formulation with the therapeutic agent and among the components. A lipid nanoparticle is a lipid particle under 200 microns in diameter. Lipid particles are generally spherical assemblies of lipids, nucleic acid, cholesterol, and stabilizing agents. Positive and negative charges, ratios, as well as hydrophilicity and hydrophobicity dictate the physical structure of the lipid particles in terms of size and orientation of components. The structural organization of these lipid particles may lead to an aqueous interior with one or more bilayers as in liposomes or it may have a solid interior as in a solid nucleic acid lipid nanoparticle. There may be phospholipid monolayers or bilayers in single or multiple forms. Lipid particles are between 1 and 1000 microns in diameter.

“Viability” when referring to cells in vitro, means the ability to continue to grow, divide, and continue to grow and divide, as is normal for the cell type or tissue culture strain. Cell viability is affected by harsh conditions or treatments. Cell viability is critical in ex vivo therapy or parenteral administration.

“Ionizable lipid.” The compositions of the invention comprise ionizable lipids as a component. As used herein, the term “ionizable lipid” refers to a lipid that is cationic or becomes ionizable (protonated) as the pH is lowered below the pKa of the ionizable group of the lipid but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH. Examples of suitable ionizable lipids are found in PCT Pub. Nos. WO20252589 and WO21000041. The ionizable lipid is present in lipid formulations according to other embodiments of the invention, preferably in a ratio of about 10 to about 40 Mol %, (“Mol %” means the percentage of the total moles that is of a particular component). The term “about” in this paragraph signifies a plus or minus range of 5 Mol % at increments of 0.1. For example, 28.7 Mol % would be in the claimed range of embodiments. DODMA, or 1,2-dioleyloxy-3-dimethylaminopropane, is an alternative ionizable lipid, as is DLin-MC3-DMA or O-(Z,Z,Z,Z-heptatriaconta-6,9,26,29-tetraen-19-yl)-4-(N,N-dimethylamino) (“MC3”). LNP may be generated from the lipid formulations including the ionizable lipids of the invention.

Phospholipids, as used herein, also known as “helper lipids” or “neutral lipids” are incorporated into lipid formulations and lipid particles of the invention in embodiments. The lipid formulations and lipid particles of the invention include one or more phospholipids at about 25 to 60 Mol % of the composition. Suitable phospholipids support the formation of particles during manufacture. Phospholipids refer to any one of several lipid species that exist in either in an anionic, uncharged or neutral zwitterionic form at physiological pH. Representative phospholipids include diacylphosphatidylcholines, diacylphosphatidylethanolamines, diacylphosphatidylglycerols, and although not strictly “phospholipids” in a technical sense, is intended to include sphingomyelins, dihydrosphingomyelins, cephalins, and cerebrosides.

Exemplary phospholipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE). In one preferred embodiment, the phospholipid is distearoylphosphatidylcholine (DSPC). In preferred embodiments, the phospholipid is DOPE. In preferred embodiments, the phospholipid is DSPC.

In another embodiment, the phospholipid is any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable phospholipids include glycolipids (e.g., monosialoganglioside GM1).

“Stabilizer” or stabilizing agent is a term used to identify the agent that is added to the ionizable lipid, the phospholipid, and the sterol that form the lipid formulation according to the invention. Examples of non-ionic stabilizing agents include: Polyethyleneglycol (PEG), Polysorbates (Tweens), TPGS (Vitamin E polyethylene glycol succinate), Brij™ S20 (polyoxyethylene (20) stearyl ether), Brij™35 (Polyoxyethylene lauryl ether, Polyethyleneglycol lauryl ether), Brij™S10 (Polyethylene glycol octadecyl ether, Polyoxyethylene (10) stearyl ether), and Myrj™52 (polyoxyethylene (40) stearate).

In some embodiments, the stabilizing agent includes PEGylated lipids including PEG-DMG 2000 (“PEG-DMG”). Other polyethylene glycol conjugated lipids may also be used. The stabilizing agent may be used alone or in combinations with each other.

In some embodiments, there is no stabilizing agent. In other embodiments, the stabilizing agent comprises about 0.1 to 3 Mol % of the overall lipid mixture. In some embodiments, the stabilizing agent includes about 0.5 to 2.5 Mol % of the overall lipid mixture. In some embodiments, the stabilizing agent is present at greater than 2.5 Mol %. In some embodiments the stabilizing agent is present at 5 Mol %. In some embodiments the stabilizing agent is present at 10 to 15 Mol %. In some embodiments, the stabilizing agent is present at 2.5 to 10 Mole %. In some embodiments, the stabilizing agent is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, and so forth. In other embodiments, the stabilizing agent is 2.6-10 Mol % of the lipid mixture. In other embodiments, the stabilizing agent is present at greater than 10 Mol % of the lipid mixture.

Sterols are included in the preferred lipid mix formulations for certain applications, and lipid particles made therefrom include cholesterol, beta-sitosterol, and 20-alpha-hydroxysterol, and phytosterol. In the lipid mixes of the invention, sterol is present at about 15 to 25 Mol % of the final lipid mix in some embodiments. In some embodiments a modified sterol or synthetically derived sterol is present.

Nucleic Acids. The lipid mix formulations and lipid particles of the present invention are useful for the systemic or local delivery of nucleic acids. In the case of vaccines, delivery is localized to the skin or muscle. As used herein, the term “nucleic acid” is meant to include any oligonucleotide or polynucleotide whose delivery into a cell causes a desirable effect. The definition includes diagnostic agents and research reagents which follow the same physical principles afforded by the invention. Fragments containing up to 50 nucleotides are generally termed oligonucleotides, and longer RNA are called polyynucleotides. In particular embodiments, oligonucleotides of the present invention are 20-50 nucleotides in length. In embodiments of the invention, polynucleotides are 996 to 4500 nucleotides in length, as in the case of messenger RNA. In particular embodiments, polynucleotides of the invention include up to 14,000 nucleotides.

The term “nucleic acid” also refers to ribonucleotides, deoxynucleotides, modified ribonucleotides, modified deoxyribonucleotides, modified phosphate-sugar-backbone oligonucleotides, other nucleotides, nucleotide analogs, and combinations thereof, and can be single stranded, double stranded, or contain portions of both double stranded and single stranded sequence, as appropriate. Messenger RNA (mRNA) can be modified or unmodified, base modified, and may include different type of capping structures, such as Cap1. In some embodiments nucleic acid refers to self-amplifying RNA (“saRNA”). In some embodiments, nucleic acid refers to a plasmid including self-amplifying RNA.

As used herein, the terms “polynucleotide” and “oligonucleotide” are used interchangeably and mean single-stranded and double-stranded polymers of nucleotide monomers, including 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or internucleotide analogs. Polynucleotides have associated counter ions, such as H+, NH4+, trialkylammonium, Mg2+, Na+, and the like. A polynucleotide may be composed entirely of deoxyribonucleotides, entirely of ribonucleotides, or chimeric mixtures thereof. Polynucleotides may include internucleotide, nucleobase and/or sugar analogs.

The term “polypeptides” herein encompasses “oligopeptides” and “proteins” and tertiary and quaternary structures thereof, that are therapeutic agents in some embodiments. An oligopeptide generally consists of from two to twenty amino acids. A polypeptide is a single linear chain of many amino acids of any length held together by amide bonds. A protein consists of one or more and may include structural proteins, energy catalysts, albumin, hemoglobin, immunoglobulins, and enzymes.

Currently, nucleic acid cargoes include deoxyribonucleic acid, complementary deoxyribonucleic acid, complete genes, ribonucleic acid, oligonucleotides and ribozymes for gene therapies targeting a variety of diseases, such as cancer, infectious diseases, genetic disorders and neurodegenerative diseases. As described herein, the nucleic acid therapeutic (NAT) is incorporated into lipid particle during its formation with compounds of the invention. More than one nucleic acid therapeutic may be incorporated in this way. They may be derived from natural sources, or more commonly, synthesized or grown in culture. Examples of nucleic acid cargo include but are not limited to antisense oligonucleotides, ribozymes, microRNA, mRNA, ribozyme, tRNA, tracrRNA, sgRNA, snRNA, siRNA, shRNA, ncRNA, miRNA, mRNA, pre-condensed DNA, plasmid or pDNA, or an aptamer. Nucleic acid reagents are used to silence genes (with for example siRNA), express genes (with for example mRNA), edit genomes (with for example CRISPR/Cas9), and reprogram cells for return to the originating organism (for example ex vivo cell therapy to reprogram immune cells for cancer therapy; autologous transfer or allogenic transfer).

The nucleic acid that is present in a lipid particle according to this invention includes any form of nucleic acid that is known. The nucleic acids used herein can be single-stranded DNA or RNA, or double-stranded DNA or RNA, or DNA-RNA hybrids. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include antisense oligonucleotides, guide RNA, including CRISPR-Cas9 gRNA, ribozymes, microRNA, mRNA, and triplex-forming oligonucleotides. More than one nucleic acid may be incorporated into the lipid particle, for example mRNA and guide RNA together, or different types of each, or in combination with protein.

In some cases, a nucleic acid encodes a genetically engineered receptor that specifically binds to a ligand, such as a recombinant receptor, and a molecule involved in a metabolic pathway, or functional portion thereof. Alternately, the molecule involved in a metabolic pathway is a recombinant molecule, including an exogenous entity. A genetically engineered receptor and the molecule involved in a metabolic pathway may be encoded by one nucleic acid or two or more different nucleic acids. In some examples, a first nucleic acid might encode a genetically engineered receptor that specifically binds to a ligand and a second nucleic acid might encode the molecule involved in a metabolic pathway.

“Therapeutic agents” as used herein include nucleic acid cargo as herein described.

The lipid particles of the invention can be assessed for size using devices that size particles in solution, such as the Malvern™ Zetasizer™. The particles generally have a mean particle diameter of from 15 nm to 1000 nm. A subgroup of lipid particles is “lipid nanoparticles” or LNP with a mean diameter of from about 15 to about 300 nm. In some embodiments, the mean particle diameter is greater than 300 nm. In some embodiments, the lipid particle has a diameter of about 300 nm or less, 250 nm or less, 200 nm or less, 150 nm or less, 100 nm or less, or 50 nm or less. In one embodiment, the lipid particle has a diameter of from about 50 to about 150 nm. Smaller particles generally exhibit increased circulatory lifetime in vivo compared to larger particles. Smaller particles have an increased ability to reach tumor sites than larger nanoparticles. In one embodiment, the lipid particle has a diameter from about 15 to about 50 nm.

The lipid particles according to embodiments of the invention can be prepared by standard T-tube mixing techniques, turbulent mixing, trituration mixing, agitation promoting orders self-assembly, or passive mixing of all the elements with self-assembly of elements into nanoparticles. A variety of methods have been developed to formulate lipid nanoparticles (LNP) containing genetic drugs. Suitable methods are disclosed in U.S. Pat. Nos. 5,753,613, 6,734,171, and 7,901,708, by way of example. These methods include mixing preformed lipid particles with nucleic acid therapeutic (NAT) in the presence of ethanol or mixing lipid dissolved in ethanol with an aqueous media containing NAT and result in lipid particles with NAT encapsulation efficiencies of 65-99%. All of these methods rely on the presence of ionizable lipid to achieve encapsulation of NAT and a stabilizing agent to inhibit aggregation and the formation of large structures. The properties of the lipid particle systems produced, including size and NAT encapsulation efficiency, are sensitive to a variety of lipid mix formulation parameters such as ionic strength, lipid and ethanol concentration, pH, NAT concentration and mixing rates.

Microfluidic two-phase droplet techniques have been applied to produce monodisperse polymeric microparticles for drug delivery or to produce large vesicles for the encapsulation of cells, proteins, or other biomolecules. The use of hydrodynamic flow focusing to create monodisperse liposomes of controlled size has also been demonstrated.

Parameters such as the relative lipid and NAT concentrations at the time of mixing, as well as the mixing rates are difficult to control using current formulation procedures, resulting in variability in the characteristics of NAT produced, both within and between preparations. This is what makes the new formulation so unique is that the ratio of ionizable lipid to phospholipid is surprisingly low. Automated micro-mixing instruments such as the NanoAssemblr® instruments (Precision NanoSystems ULC, Vancouver, Canada) enable the rapid and controlled manufacture of nanomedicines (liposomes, lipid nanoparticles, and polymeric nanoparticles). NanoAssemblr® instruments accomplish controlled molecular self-assembly of nanoparticles via microfluidic mixing cartridges that allow millisecond mixing of nanoparticle components at the nanoliter, microlitre, or larger scale with customization or parallelization. Rapid mixing on a small scale allows reproducible control over particle synthesis and quality that is not possible in larger instruments.

Preferred methods incorporate instruments such as the microfluidic mixing devices like the NanoAssemblr® Spark™, Ignite™, Benchtop™ and NanoAssemblr® Blaze™ in order to achieve nearly 100% of the nucleic acid used in the formation process is encapsulated in the particles in one step. In preferred embodiments, the lipid particles are prepared by a process by which from about 75 to about 100% of the nucleic acid used in the formation process is encapsulated in the particles.

U.S. Pat. Nos. 9,758,795 and 9,943,846 describe methods of using small volume mixing technology and novel formulations derived thereby. U.S. Pat. No. 10,159,652 describes more advanced methods of using small volume mixing technology and products to formulate different materials. U.S. Pat. No. 9,943,846 discloses microfluidic mixers with different paths and wells to elements to be mixed. PCT Pub. No. WO 2017117647 discloses microfluidic mixers with disposable sterile paths. U.S. Pat. No. 10,076,730 discloses bifurcating toroidal micromixing geometries and their application to microfluidic mixing. PCT Pub. No. WO2018006166 discloses a programmable automated micromixer and mixing chips therefore. U.S. Design Nos. D771834, D771833, D772427, D803416, D800335, D800336 and D812242 disclose mixing cartridges having microchannels and mixing geometries for mixer instruments sold by Precision NanoSystems ULC.

In embodiments of the invention, devices for biological microfluidic mixing are used to prepare the lipid particles according to embodiments of the invention. The devices include a first and second stream of reagents, which feed into the microfluidic mixer, and lipid particles are collected from the outlet, or emerge into a sterile environment.

The first stream includes a therapeutic agent in a first solvent. Suitable first solvents include solvents in which the therapeutic agents are soluble and that are miscible with the second solvent. Suitable first solvents include aqueous buffers. Representative first solvents include citrate and acetate buffers, or optionally other low pH buffers.

The second stream includes lipid mix materials in a second solvent. Suitable second solvents include solvents in which the ionizable lipids according to embodiments of the invention are soluble, and that are miscible with the first solvent. Suitable second solvents include 1,4-dioxane, tetrahydrofuran, acetone, acetonitrile, dimethyl sulfoxide, dimethylformamide, acids, and alcohols. Representative second solvents include aqueous ethanol 90%, or anhydrous ethanol.

In one embodiment of the invention, a suitable device includes one or more microchannels (i.e., a channel having its greatest dimension less than 2 millimeters). In one example, the microchannel has a diameter from about 20 to about 300 μm. In another example, the microchannel has a diameter from about 300 to about 1000 μm In examples, at least one region of the microchannel has a principal flow direction and one or more surfaces having at least one groove or protrusion defined therein, the groove or protrusion having an orientation that forms an angle with the principal direction (e.g., a staggered herringbone mixer), as described in U.S. Pat. No. 9,943,846, or a bifurcating toroidal flow as described in U.S. Pat. No. 10,076,730. To achieve maximal mixing rates, it is advantageous to avoid undue fluidic resistance prior to the mixing region. Thus, one example of a device has non-microfluidic channels having dimensions greater than 1000 μm, to deliver the fluids to a single mixing channel.

Less complex mixing methods and instruments such as those disclosed in, for example, U.S. Published Patent Application No. 20040262223, are also useful in creating lipid particle formulations of the invention.

The lipid mixes of the present invention may be used to deliver a therapeutic agent to a cell, in vitro or in vivo. In particular embodiments, the therapeutic agent is a nucleic acid, which is delivered to a cell using nucleic acid-lipid particles of the present invention. The nucleic acid can be an siRNA, miRNA, an LNA, a plasmid, replicon (including a vector with antigenic mRNA), a self-amplifying RNA, an mRNA, a guide RNA, a transposon, or a single gene.

In other embodiments, the therapeutic agent is an oligopeptide, polypeptide, or protein which is delivered to a cell using peptide-lipid particles of the present invention. In other embodiments, the therapeutic agent is a mixture of nucleic acid and protein components, such as Cas9. The methods and lipid mix formulations may be readily adapted for the delivery of any suitable therapeutic agent for the treatment of any disease or disorder that would benefit from such treatment.

In certain embodiments, the present invention provides methods for introducing a nucleic acid into a cell (i.e., transfection). Transfection is a technique commonly used in molecular biology for the introduction of nucleic acid cargo (or NATs) from the extracellular to the intracellular space for the purpose of transcription, translation and expression of the delivered nucleic acid therapeutic (NAT) for production of some gene product or for down regulating the expression of a disease-related gene. Transfection efficiency is commonly defined as either the i) percentage of cells in the total treated population showing positive expression of the delivered gene, as measured by live or fixed cell imaging (for detection of fluorescent protein), and flow cytometry or ii) the intensity or amount of protein expressed by treated cell(s) as analyzed by live or fixed cell imaging or flow cytometry or iii) using protein quantification techniques such as ELISA, or western blot. These methods may be carried out by contacting the lipid particles or lipid mix formulations of the present invention with the cells for a period of time sufficient for intracellular delivery to occur.

Typical applications include using well known procedures to provide intracellular delivery of siRNA to knock down or silence specific cellular targets in vitro and in vivo. Alternatively, applications include delivery of DNA or mRNA sequences that code for therapeutically useful polypeptides. In this manner, therapy is provided for genetic diseases by supplying deficient or absent gene products. Methods of the present invention may be practiced in vitro, ex vivo, or in vivo. For example, the lipid mix formulations of the present invention can also be used for delivery of nucleic acids to cells in vivo, using methods which are known to those of skill in the art. In another example, the lipid mix formulations of the invention can be used for delivery of nucleic acids to a sample of patient cells that are ex vivo, then are returned to the patient.

The delivery of nucleic acid cargo by a lipid particle of the invention is described below.

For in vivo administration, the pharmaceutical compositions are preferably administered parenterally (e.g., intraarticularly, intravenously, intraperitoneally, subcutaneously, intrathecally, intradermally, intratracheally, intraosseous, intramuscularly or intratumorally). In particular embodiments, the pharmaceutical compositions are administered intravenously, intramuscularly, intrathecally, or intraperitoneally by a bolus injection. Other routes of administration include topical (skin, eyes, mucus membranes), oral, pulmonary, intranasal, sublingual, rectal, and vaginal.

For ex vivo applications, the pharmaceutical compositions are preferably administered to biological samples that have been removed from the organism, then the cells are washed and restored to the organism. The organism may be a mammal, and in particular may be human. This process is used for cell reprogramming, genetic restoration, or. immunotherapy, for example.

In one embodiment, the present invention provides a method of modulating the expression of a target polynucleotide or polypeptide. These methods generally comprise contacting a cell with a lipid particle of the present invention that is associated with a nucleic acid capable of modulating the expression of a target polynucleotide or polypeptide. As used herein, the term “modulating” refers to altering the expression of a target polynucleotide or polypeptide. Modulating can mean increasing or enhancing, or it can mean decreasing or reducing.

In related embodiments, the present invention provides a method of treating a disease or disorder characterized by overexpression of a polypeptide in a subject, comprising providing to the subject a pharmaceutical composition of the present invention, wherein the therapeutic agent is selected from an siRNA, a microRNA, an antisense oligonucleotide, and a plasmid capable of expressing an siRNA, a microRNA, or an antisense oligonucleotide, and wherein the siRNA, microRNA, or antisense RNA includes a polynucleotide that specifically binds to a polynucleotide that encodes the polypeptide, or a complement thereof.

In related embodiments, the present invention provides a method of treating a disease or disorder characterized by under-expression of a polypeptide in a subject, comprising providing to the subject a pharmaceutical composition of the present invention, wherein the therapeutic agent is selected from an mRNA, a self-amplifying RNA (saRNA), or a plasmid, includes a nucleic acid therapeutic that specifically encodes or expresses the under-expressed polypeptide, or a complement thereof. Examples include RNA vaccines, and more particularly self-amplifying mRNA vaccines.

Methods of delivery of biological active agents for treatment of disease include, in one embodiment, the compounds, compositions, methods and uses of the invention are for delivering a biologically active agent to liver cells (e.g., hepatocytes). In one embodiment, the compounds, compositions, methods and uses of the invention are for delivering a biologically active agent to a tumor or to tumor cells (e.g., a primary tumor or metastatic cancer cells). In another embodiment, the compounds, compositions, methods and uses are for delivering a biologically active agent to the skin adipose, muscle and lymph nodes (subcutaneous dosing).

For delivery of a biologically active agent to the liver or liver cells, in one embodiment a formulation of the invention is contacted with the liver or liver cells of the via parenteral administration (e.g., intravenous, intramuscular, subcutaneous administration) or local administration (e.g., direct injection, portal vein injection, catheterization, stenting), to facilitate delivery. For delivery of a biologically active agent to the kidney or kidney cells, in one embodiment a formulation of the invention is contacted with the kidney or kidney cells of the patient via parenteral administration (e.g., intravenous, intramuscular, subcutaneous administration) or local administration (e.g., direct injection, catheterization, stenting), to facilitate delivery. For delivery of a biologically active agent to a tumor or tumor cells, in one embodiment, a formulation of the invention is contacted with the tumor or tumor cells of the patient via parenteral administration (e.g., intravenous, intramuscular, subcutaneous administration) or local administration (e.g., direct injection, catheterization, stenting), to facilitate delivery.

For delivery of a biologically active agent to the CNS or CNS cells), in one embodiment, a formulation of the invention is contacted with the CNS or CNS cells (e.g., brain cells and/or spinal cord cells) of the patient via parenteral administration (e.g., intravenous, intramuscular, subcutaneous administration) or local administration (e.g., direct injection, catheterization, stenting, osmotic pump administration (e.g., intrathecal or ventricular)), to facilitate delivery. For delivery of a biologically active agent to the Peripheral Nervous System (PNS) or PNS cells, in one embodiment a formulation of the invention is contacted with the PNS or PNS cells of the patient via parenteral administration (e.g., intravenous, intramuscular, subcutaneous administration) or local administration (e.g., direct injection), to facilitate delivery. For delivery of a biologically active agent to a lung or lung cells, in one embodiment a formulation of the invention is contacted with the lung or lung cells of the patient via parenteral administration (e.g., intravenous, intramuscular, subcutaneous administration) or local administration (e.g., pulmonary administration directly to lung tissues and cells), to facilitate delivery.

For delivery of a biologically active agent to the vasculature or vascular cells, in one embodiment, a formulation of the invention is contacted with the vasculature or vascular cells of the patient via parenteral administration (e.g., intravenous, intramuscular, subcutaneous administration) or local administration (e.g., clamping, catheterization, stenting), to facilitate delivery.

For delivery of a biologically active agent to the skin or skin cells (e.g., dermis cells and/or follicular cells), in one embodiment, a formulation of the invention is contacted with the skin or skin cells (e.g., dermis cells and/or follicular cells) of the patient via parenteral administration (e.g., intravenous, intramuscular, subcutaneous administration) or local administration (e.g., direct dermal application, iontophoresis), to facilitate delivery. For delivery of a biologically active agent to an eye or ocular cells (e.g., macula, fovea, cornea, retina), in one embodiment a formulation of the invention is contacted with the eye or ocular cells (e.g., macula, fovea, cornea, retina) of the patient via parenteral administration (e.g., intravenous, intramuscular, subcutaneous administration) or local administration (e.g., direct injection, intraocular injection, periocular injection, subretinal, iontophoresis, use of eyedrops, implants), to facilitate delivery. For delivery of a biologically active agent to an ear or cells of the ear (e.g., cells of the inner ear, middle ear and/or outer ear), in one embodiment formulation of the invention is contacted with the ear or cells of the ear (e.g., cells of the inner ear, middle ear and/or outer ear) of the patient as is generally known in the art, such as via parenteral administration (e.g., intravenous, intramuscular, subcutaneous administration) or local administration (e.g., direct injection), to facilitate delivery. For delivery of a biologically active agent (e.g., RNA encoding an immunogen) to cells of the immune system (e.g., antigen-presenting cells, including professional antigen presenting cells), in one embodiment formulation of the invention is delivered intramuscularly, after which immune cells can infiltrate the delivery site and process delivered RNA and/or process encoded antigen produced by non-immune cells, such as muscle cells. Such immune cells can include macrophages (e.g., bone marrow derived macrophages), dendritic cells (e.g., bone marrow derived plasmacytoid dendritic cells and/or bone marrow derived myeloid dendritic cells), T-cells, and monocytes (e.g., human peripheral blood monocytes), etc. (for example, see WO2012/006372).

Immunization. For immunization purposes, a formulation of the invention will generally be prepared as an injectable, a pulmonary or nasal aerosol, or in a delivery device (e.g., syringe, nebulizer, sprayer, inhaler, dermal patch, etc.). This delivery device can be used to administer a pharmaceutical formulation to a subject, e.g., to a human, for immunization.

According to the invention, for immunization purposes, in some embodiments, the invention encompasses delivering an RNA that encodes an immunogen. This immunogen elicits an immune response which recognizes the immunogen, to provide immunity against a pathogen, or against an allergen, or against a tumor antigen. Immunizing against disease and/or infection caused by a pathogen is preferred.

The RNA is delivered with a lipid formulation of the invention (e.g., formulated as a liposome or LNP). In some embodiments, the invention utilizes LNPs within which immunogen-encoding RNA is encapsulated. Encapsulation within LNPs can protect RNA from RNase digestion. The encapsulation efficiency does not have to be 100%. Presence of external RNA molecules (e.g., on the exterior surface of a liposome or LNP) or “naked” RNA molecules (RNA molecules not associated with a liposome or LNP) is acceptable. Preferably, for a formulation comprising lipids and RNA molecules, at least half of the RNA molecules (e.g., at least 50%, at least 60%, at least 65%, at least 70%, at least 75%, at least 80%, at least 85%, at least 90%, at least 95%, at least about 96%, at least about 97%, at least about 98%, or at least 99% of the RNA molecules) are encapsulated in LNPs or complexed with LNPs.

Some lipid nanoparticles may comprise a lipid core (e.g., the formulation may comprise a mixture of LNPs and nanoparticles with a lipid core). In such cases, the RNA molecules may be encapsulated by LNPs that have an aqueous core or cores, and complexed with the LNPs that have a lipid core by noncovalent interactions (e.g., ionic interactions between negatively charged RNA and cationic lipid). Encapsulation and complexation with LNPs (whether with a lipid or aqueous core) can protect RNA from RNase digestion. The encapsulation/complexation efficiency does not have to be 100%. Presence of “naked” RNA molecules (RNA molecules not associated with the LNP) is acceptable. Preferably, for a formulation comprising a population of LNPs and a population of RNA molecules, at least half of the population of RNA molecules (e.g., at least e.g., at least 50%, at least 55%, at least 60%, at least 65%, at least 70%, at least 75%, at least 80%, at least 85%, at least 90%, or at least 95% of the RNA molecules) are either encapsulated in LNPs, or complexed with LNPs.

Some lipid nanoparticles have multilamellar components.

For delivery of immunogen-coding RNA, the preferred range of LNP diameters is in the range of 60-180 nm, and in more particular embodiments, in the range of 80-160 nm. An LNP can be part of a composition comprising a population of LNPS, and the LNPS within the population can have a range of diameters. For a composition comprising a population of LNPs with different diameters, it is preferred that (i) at least 80% by number of the LNP have diameters in the range of 60-180 nm, e.g., in the range of 80-160 nm, (ii) the average diameter (by intensity, e.g., Z-average) of the population is ideally in the range of 60-180 nm, e.g., in the range of 80-160 nm; and/or the diameters within the plurality have a polydispersity index <0.2. To obtain LNPs with the desired diameter(s), mixing can be performed using a process in which two feed streams of aqueous RNA solution are combined in a single mixing zone with one stream of an ethanolic lipid solution, all at the same flow rate e.g., in a microfluidic channel. See other description relating to NanoAssemblr® microfluidic mixers sold by Precision NanoSystems ULC, Vancouver, Canada.

RNA Molecules. After in vivo administration of an immunization composition (“vaccine vector LNP”), the delivered RNA is released and is translated inside a cell to provide the immunogen in situ. In certain embodiments, the RNA is plus (“+”) stranded, so it can be translated by cells without needing any intervening replication steps such as reverse transcription. In certain embodiments, the RNA is a self-replicating RNA. A self-replicating RNA molecule (replicon) can, when delivered to a vertebrate cell even without any proteins, lead to the production of multiple daughter RNAs by transcription from itself (via an antisense copy which it generates from itself). A self-replicating RNA molecule is thus in certain embodiments: a (+) strand molecule that can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded immunogen, or may be transcribed to provide further transcripts with the same sense as the delivered RNA which are translated to provide in situ expression of the immunogen. The overall result of this sequence of transcriptions is an amplification in the number of the introduced replicon RNAs and so the encoded immunogen becomes a major polypeptide product of the host cells.

One suitable system for achieving self-replication is to use an alphavirus-based RNA replicon. These (+) stranded replicons are translated after delivery to a cell to yield a replicase (or replicase-transcriptase). The replicase is translated as a polyprotein which auto cleaves to provide a replication complex which creates genomic (−) strand copies of the (+) strand delivered RNA. These (−) strand transcripts can themselves be transcribed to give further copies of the (+) stranded parent RNA, and also to give a subgenomic transcript which encodes the immunogen. Translation of the subgenomic transcript thus leads to in situ expression of the immunogen by the infected cell. Suitable alphavirus replicons can use a replicase from a Sindbis virus, a Semliki Forest virus, an eastern equine encephalitis virus, or more preferably, a Venezuelan equine encephalitis virus, etc. The system may be a hybrid or chimeric replicase in some embodiments. A preferred embodiment is a replicon according to one embodiment of the invention, showing a PNI-V101 replicon capable of self-amplifying in mammalian cells and expressing, through mRNA assembled, immunogenic proteins such as Sars-COV-2 spike proteins.

An RNA molecule may have a 5′ cap (e.g., a 7-methylguanosine). This cap can enhance in vivo translation of the RNA. The 5′ nucleotide of an RNA molecule useful with the invention may have a 5′ triphosphate group. In a capped RNA, this may be linked to a 7-methylguanosine via a 5′-to-5′ bridge. A 5′ triphosphate can enhance RIG-I binding and thus promote adjuvant effects. An RNA molecule may have a 3′ poly A tail. It may also include a poly A polymerase recognition sequence (e.g., AAUAAA) near its 3′ end. An RNA molecule useful with the invention for immunization purposes will typically be single-stranded. Single-stranded RNAs can generally initiate an adjuvant effect by binding to TLR7, TLR8, RNA helicases and/or PKR.

RNA molecules can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). As discussed in WO2011/005799, the self-replicating RNA can include (in addition to any 5′ cap structure) one or more nucleotides having a modified nucleobase. For instance, a self-replicating RNA can include one or more modified pyrimidine nucleobases, such as pseudouridine and/or 5 methylcytosine residues. In some embodiments, however, the RNA includes no modified nucleobases, and may include no modified nucleotides, i.e., all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first I, 2 or 3 5′ ribonucleotides may be methylated at the 2′ position of the ribose. An RNA used with the invention for immunization purposes ideally includes only phosphodiester linkages between nucleosides, but in some embodiments, it contains phosphoramidate, phosphorothioate, and/or methylphosphonate linkages. The invention includes embodiments in which multiple species of RNAs are formulated with a lipid formulation provided by the invention, such as two, three, four or more species of RNA, including different classes of RNA (such as mRNA, siRNA, self-replicating RNAs, and combinations thereof).

Immunogen RNA molecules used with the invention for immunization purposes, in some embodiments, encode a polypeptide immunogen. In these embodiments, after administration, the RNA is translated in vivo and the immunogen can elicit an immune response in the recipient. The immunogen may elicit an immune response against a pathogen (e.g., a bacterium, a virus, a fungus or a parasite) but, in some embodiments, it elicits an immune response against an allergen or a tumor antigen. The immune response may comprise an antibody response (usually including IgG) and/or a cell mediated immune response. The polypeptide immunogen will typically elicit an immune response which recognizes the corresponding pathogen (or allergen or tumor) polypeptide, but in some embodiments the polypeptide may act as a mimotope to elicit an immune response which recognizes a saccharide. The immunogen will typically be a surface polypeptide, e.g., an adhesin, a hemagglutinin, an envelope glycoprotein, a spike glycoprotein, etc. The RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from a replicon, then one or more of these may be provided with an upstream IRES or an additional viral promoter element. Alternatively, multiple immunogens may be expressed from a polyprotein that encodes individual immunogens fused to a short autocatalytic protease (e.g., foot-and-mouth disease virus 2A protein), or as inteins. In certain embodiments, polypeptide immunogens (e.g., I, 2, 3, 4, 5, 6, 7, 8, 9, 10, or more immunogens) may be used, either alone or together with a RNA molecule, such as a self-replicating RNA, encoding one or more immunogens (either the same or different as the polypeptide immunogens).

In some embodiments, the immunogen elicits an immune response against Coronavirus spp., whose immunogens include, but are not limited to, those derived from a SARS CoV-1, SARS-CoV-2(12); human influenza virus, and Neisseria meningitidis for which useful immunogens include, but are not limited to, membrane proteins such as adhesins, autotransporters, toxins, iron acquisition proteins, and factor H binding protein. A combination of three useful polypeptides is disclosed in Giuliani et al. (Proc Natl Acad Sci USA. 2006; 103(29): 10834-9. Epub 2006 Jul. 6. doi: 10.1073/pnas.0603940103. PubMed PMID: 16825336; PubMed Central PMCID: PMC2047628); Streptococcus pneumoniae, for which useful polypeptide immunogens are disclosed in WO2009/016515 including the RrgB pilus subunit, the beta-N-acetyl-hexosaminidase precursor (spr0057), spr0096, general stress protein GSP-781 (spr2021, SP2216), serine/threonine kinase StkP (SP1732), and pneumococcal surface adhesin PsaA;

Hepatitis viruses, whose immunogens can include hepatitis B virus surface antigen (HBsAg), hepatitis C virus, delta hepatitis virus, hepatitis E virus, or hepatitis G virus antigens; Rhabdovirus: immunogens include, but are not limited to, those derived from a Rhabdovirus, such as a Lyssavirus (e.g., a Rabies virus) and Vesiculovirus (VSV); Caliciviridae, whose immunogens include, but are not limited to, those derived from Calciviridae, such as Norwalk virus (Norovirus), and Norwalk-like Viruses, such as Hawaii Virus and Snow Mountain Virus; avian infectious bronchitis (IBV), Mouse hepatitis virus (MHV), and Porcine transmissible gastroenteritis virus (TGEV); Retrovirus, whose immunogens include those derived from an Oncovirus, a Lentivirus (e.g., HIV-I or HIV-2) or a Spumavirus; Reovirus: immunogens include, but are not limited to, those derived from an Orthoreovirus, a Rotavirus, an Orbivirus, or a Coltivirus; Parvovirus, whose immunogens include those derived from Parvovirus B19; Herpesvirus, whose immunogens include those derived from a human herpesvirus, such as Herpes Simplex Viruses (HSV) (e.g., HSV types I and 2), Varicella-zoster virus (VZV), Epstein Barr virus (EBV), Cytomegalovirus (CMV), Human Herpesvirus 6 (HHV6), Human Herpesvirus 7 (HHV7), and Human Herpesvirus 8 (HHV8); Papovaviruses, whose immunogens include those derived from Papillomaviruses and Adenovirus.

In some embodiments, the immunogen elicits an immune response to Chikungunya virus; in other embodiments, the immunogen elicits an immune response to Zika virus.

In some embodiments, the immunogen elicits an immune response against a virus which infects fish.

Fungal immunogens may be derived from Dermatophytres and other opportunistic organisms.

In some embodiments, the immunogen elicits an immune response against a parasite from the Plasmodium genus, such as P. falciparum, P. vivax, P. malariae or P. ovale. Thus the invention may be used for immunizing against malaria. In some embodiments the immunogen elicits an immune response against a parasite from the Caligidae family, particularly those from the Lepeophtheirus and Caligus genera e.g., sea lice such as Lepeophtheirus salmonis or Caligus rogercresseyi.

In some embodiments, the immunogen is an mRNA specific to neoantigens in cancer cells or solid tumours.

In some embodiments, the immunogen is a tumor antigen selected from: (a) cancer-testis antigens such as NY-ESO-I, SSX2, SCPI as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUMI (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLRFUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT I (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-I (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte antigens such as MART-1/Melan A, gp100, MCIR, melanocyte-stimulating hormone receptor, tyrosinase, tyrosinase related protein-I/TRPI and tyrosinase related protein-2/TRP2 (associated with, e.g., melanoma); (e) prostate associated antigens such as PAP, PSA, PSMA, PSH-PI, PSM-PI, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23HI, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29&BCAA), CA 195, CA 242, CA-50, CAM43, CD68&KPI, CO-029, FGF-5, Ga733 (EpCAM), HTgp-175, M344, MA-50, MG7-Ag, MOV18, NB/70K, NY-CO-I, RCASI, SDCCAG16, TA-90 (Mac-2 binding protein/cyclophilin C-associated protein), TAAL6, TAG72, TLP, TPS, and the like.

Pharmaceutical Compositions for Vaccines. A pharmaceutical composition of the invention, particularly one useful for immunization, may include one or more small molecule immunopotentiators. Pharmaceutical compositions of the invention may include one or more preservatives, such as thiomersal or 2 phenoxyethanol. Mercury-free and preservative-free vaccines can be prepared.

Compositions comprise an effective amount of the lipid formulations described herein (e.g., LNP), as well as any other components, as needed. Immunologically effective amount refers to the amount administered to an individual, either in a single dose or as part of a series, is effective for treatment (e.g., prophylactic immune response against a pathogen). This amount varies depending upon the health and physical condition of the individual to be treated, age, the taxonomic group of individual to be treated (e.g., non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The LNP-formulated RNA and pharmaceutical compositions described herein are for in vivo use for inducing an immune response against an immunogen of interest. The invention provides a method for inducing an immune response in a vertebrate comprising administering an effective amount of the LNP formulated RNA, or pharmaceutical composition, as described herein. The immune response is preferably protective and preferably involves antibodies and/or cell-mediated immunity. The compositions may be used for both priming and boosting purposes. Alternatively, a prime-boost immunization schedule can be a mix of RNA and the corresponding polypeptide immunogen (e.g., RNA prime, protein boost).

The invention also provides an LNP or pharmaceutical composition thereof for use in inducing an immune response in a vertebrate. The invention also provides the use of a LNP or pharmaceutical composition thereof in the manufacture of a medicament for inducing an immune response in a vertebrate. By inducing an immune response in the vertebrate by these uses and methods, the vertebrate can be protected against various diseases and/or infections e.g., against bacterial and/or viral diseases as discussed above. Vaccines according to the invention may either be prophylactic (i.e., to prevent infection) or therapeutic (i.e. to treat infection), but will typically be prophylactic. The vertebrate is preferably a mammal, such as a human or a large veterinary mammal (e.g., horses, cattle, deer, sheep, llamas, goats, pigs).

Compositions of the invention will generally be administered directly to a patient. Direct delivery may be accomplished by parenteral injection (e.g., subcutaneously, intraperitoneally, intravenously, intramuscularly, intradermally, or to the interstitial space of a tissue. Alternative delivery routes include rectal, oral (e.g., tablet, drops, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g., a hypodermic needle), but needle-free injection may alternatively be used. A typical intramuscular dose is 0.5 ml. The invention may be used to induce systemic and/or mucosal immunity, preferably to elicit an enhanced systemic and/or mucosal immunity. Dosage can be by a single dose schedule or a multiple dose schedule. Multiple doses may be used in a primary immunization schedule and/or in a booster immunization schedule.

In a multiple dose schedule, the various doses may be given by the same or different routes, for example, a parenteral prime and mucosal boost, a mucosal prime and parenteral boost, etc. Multiple doses will typically be administered at least one week apart (e.g., about two weeks, about three weeks, about four weeks, about six weeks, about eight weeks, about ten weeks, about 12 weeks, about 16 weeks, etc.). In one embodiment, multiple doses may be administered approximately six weeks, ten weeks and 14 weeks after birth, e.g., at an age of six weeks, ten weeks and 14 weeks, as often used in the World Health Organization's Expanded Program on Immunization (“EPI”). In an alternative embodiment, two primary doses are administered about two months apart, e.g., about seven, eight or nine weeks apart, followed by one or more booster doses about six months to one year after the second primary dose, e.g., about six, eight, ten or 12 months after the second primary dose. In a further embodiment, three primary doses are administered about two months apart, e.g., about seven, eight or nine weeks apart, followed by one or more booster doses about six months to one year after the third primary dose.

In embodiments, the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams and Wilkins, Baltimore, MD, 2006). The use of a conventional excipient medium is contemplated herein, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid particles may be increased and/or decreased. The change in particle size may be able to help counter biological reaction such as, but not limited to, inflammation or may increase the biological effect of the NAT delivered to mammals by changing biodistribution. Size may also be used to determine target tissue, with larger particles being cleared quickly and smaller one reaching different organ systems.

Pharmaceutically acceptable excipients used in the manufacture of pharmaceutical compositions including the LNP include, but are not limited to, inert diluents, surface active agents and/or emulsifiers, preservatives, buffering agents, lubricating agents, and/or oils. Such excipients may optionally be included in the pharmaceutical formulations of the invention.

The following is a description of representative lipid particles prepared with nucleic acid (LNP), how they are made, evidence of their advantages, and methods for using them to deliver therapeutic benefits.

### EXAMPLES

General considerations: All solvents and reagents were commercial products and used as such unless noted otherwise. Temperatures are given in degrees Celsius.

### ABBREVIATIONS

EPO=erythropoietin
GFP=green fluorescent protein
ug=μg=microgram
pg=picogram
ng=nanogram
g=gram
h=hour(s)
HPLC=High performance liquid chromatography
MFI=Median Fluorescence Intensity
min=minute(s)
mL=milliliter(s)
mmol=millimole(s)
N/P ratio=the ratio of positively-chargeable polymer amine (N=nitrogen)
groups to negatively-charged nucleic acid phosphate (P) groups
PBS=phosphate buffered solution
wt=weight
Deg. C=Degree Celsius

“Gene of interest” (GOI) signifies a genetic element or elements intended for expression to achieve a therapeutic goal, including immunization. A5 SARS Cov-2 antigenic coding elements and epidermal growth factor (EPO) are examples of a GOI to illustrate the present invention, but GOI is not limited to this demonstrated example.

IL=ionizable lipid, a lipid that is cationic at higher pH, and converts to uncharged at lower pH. IL are commonly used in formulations of nucleic acid cargo.

Stabilizing Agent=any stabilizing agent including polyethyleneglycol derivatives, including PEG-DMG 2000 and other suitable polymers, which have the purpose of extending circulation life, among other things.

Components of the Lipid Mixes include the ionizable lipid, phospholipid, cholesterol, and stabilizing agent. Low pH buffers (3-6) may be used. For ionizable aminolipids, the pH of the buffer is typically below the pKa of the lipid.

PNI 516 is ionizable lipid (Z)-3-(2-((1,17-bis(2-octylcyclopropyl)heptadecan-9-yl)oxy)-2-oxoethyl)-2-(pent-2-en-1-yl)cyclopentyl 4-(dimethylamino)butanoate found in PCT Publication WO20252589 A1 by Jain, N, Thomas A, and Brown A.

PNI 560 is ionizable lipid (Z)-3-(2-((1,17-bis(2-octylcyclopropyl)heptadecan-9-yl)oxy)-2-oxoethyl)-2-(pent-2-en-1-yl)cyclopentyl 1,4-dimethylpiperidine-4-carboxylate found in PCT Publication WO20252589 by Jain et al.

PNI 127 is an ionizable lipid found in PCT Publication WO21000041 A1 by Thomas, A; Jain, N; and Brown A. Its structural formula is (2R,3S,4S)-2-(((1,4-dimethylpiperidine-4-carbonyl)oxy)methyl)tetrahydrofuran-3,4-diyl (9Z,9′Z,12Z,12′Z)-bis(octadeca-9,12-dienoate).

Also found in PCT Publication WO2100041 by Thomas et al. are:

PNI 550, 3-(2-((1,17-bis(2-octylcyclopropyl)heptadecan-9-yl)oxy)-2-oxoethyl)cyclopentyl 4-(dimethylamino)butanoate;

PNI 580: (2R,3S,4S)-2-(((4-(dimethylamino)butanoyl)oxy)methyl)tetrahydrofuran-3,4-diyl bis(2-hexyldecanoate); PNI 659: ((2R,3R,4S)-3,4-bis((2-hexyldecyl)oxy)tetrahydrofuran-2-yl)methyl 4-(dimethylamino)butanoate; PNI 721: (2R,3S,4S)-2-((((2-(dimethylamino)ethyl)carbamoyl)oxy)methyl)tetrahydrofuran-3,4-diyl bis(2-hexyldecanoate); PNI 722: 2-(((2R,3R,4S)-3,4-bis((2-hexyldecyl)oxy)tetrahydrofuran-2-yl)methoxy)-N,N-dimethylethan-1-amine; PNI 726: (2R,3S,4S)-2-((3-(dimethylamino)propoxy)methyl)tetrahydrofuran-3,4-diyl bis(2-hexyldecanoate); PNI 728: ((2R,3R,4S)-3,4-bis((2-hexyldecyl)oxy)tetrahydrofuran-2-yl)methyl(2-(dimethylamino)ethyl)carbamate; and PNI 730: (2R,3S,4S)-2-((2-(dimethylamino)ethoxy)methyl)tetrahydrofuran-3,4-diyl bis(2-hexyldecanoate).

### Example 1

Method for Self-Amplifying mRNA Synthesis.

The restriction digestion of a circular plasmid encoding SARS Covid spike protein was carried out according to manufacturer's instructions for BspQI (New England BioLabs Inc., Catalog number R0712S) or PmeI (New England BioLabs Inc., Catalog number R0560S), in vendor-prescribed buffers.

The linearized vector was purified using Phenol/Chloroform/Isoamyl alcohol (25:24:1) and sodium acetate precipitation. Briefly, equal volumes of Phenol/Chloroform/Isoamyl alcohol solution were added to the linearized vector, vortexed for 20 seconds and incubated at room temperature for 2 minutes. The mixture was spun, after which the top aqueous phase containing the linearized vector was carefully pipetted into a clean RNase/Dnase free tube and precipitated 3 volumes of 100% ethanol were added, mixed well and removed carefully and the DNA pellet air dried and resuspended in nuclease free water. The concentration and purity of the linearized vector was checked using NanoDrop™ spectrophotometer (VWR).

In vitro transcription was carried out using HiScribe™ T7 High Yield RNA Synthesis Kit (New England BioLabs, Inc., Catalog number E2040S) followed by linear DNA template digestion, performed using TURBO™ DNase (Thermofisher Scientific, Catalog number AM2238), and the final in vitro transcribed self-amplifying RNA (saRNA) was capped using Vaccina Capping System™ (New England Biolabs Inc, Catalog number M2080S). All these processes were performed according to the manufacturer's protocol to generate the self-amplifying RNA utilizing the DNA templates obtained from the vector linearization strategy. The purification of the capped saRNA was performed using standard salt precipitation, followed by 70% ethanol wash and resuspension of the RNA pellet in RNA storage solution (Thermofisher). This is described in PCT Pub. No. WO23057979 by Geall et al.

### Example 2

Microfluidic Mixing of Nucleic Acid into Lipid Nanoparticles (LNP)

Lipid mix formulation of lipid particles were generated by rapidly mixing lipid-ethanol solution with an aqueous buffer inside a microfluidic mixer designed to induce chaotic advection and provide a controlled mixing environment at intermediate Reynolds number (24<Re<1000). The microfluidic channels have herringbone features or are configured in a manner as shown in PCT Pub. No. WO2017117647 or U.S. Pat. No. 10,835,878.

Particle sizes and “polydispersity index” (PDI) of the lipid particle were measured by dynamic light scattering (DLS). PDI indicates the width of the particle distribution. This is a parameter calculated from a cumulative analysis of the (DLS)-measured intensity autocorrelation function assuming a single particle size mode and a single exponential fit to the autocorrelation function. From a biophysical point of view, a PDI below 0.1 indicates that the sample is monodisperse. The particles produced by mechanical micromixers such as the NanoAssemblr® Spark™ and NanoAssemblr® Ignite™ (Precision NanoSystems ULC) are substantially homogeneous in size assuming all other variables are neutral. A lower PDI indicates a more homogenous population of lipid particles. The Spark™ instrument is used in a screening setting to identify the lead compositions. Once the composition is selected, the lipid particle can be fine-tuned using the NanoAssemblr® Ignite™ instrument. Once the process parameters Flow Rate Ratio and Total Flow Rate are identified for a specific nanoparticle formulation, the nanoparticle technology can be scaled up using the same process parameter values.

Self-amplifying RNA or nucleic acid therapeutic (NAT) as described above, was diluted using sodium acetate buffer to the required concentration. Lipid nucleic acid particle (LNP) samples were then prepared as described by running both fluids using the NanoAssemblr® Ignite instrument. Briefly, 63 μg of nucleic acids in sodium acetate buffer in a total volume of 0.75 mL was mixed with 0.25 mL of 12.5 mM lipid mix solution as required by the N/P ratio of 8, then LNP were diluted by in line dilution at 2:1 ratio in PBS.

The resulting lipid nucleic acid particles (LNP) made were immediately diluted down with 48 μL Ca++ and Mg++ free 1×PBS at pH 7.4 in the aqueous output well. These LNP were immediately collected into microcentrifuge tubes containing 96 μL of the same buffer at pH 7.4. Encapsulation efficiency was measured by a modified Ribogreen™ assay (Quanti-iT RiboGreen™ RNA assay kit, Fisher). This information was used to establish the desired dosage.

Lipid particles were also manufactured by a larger microfluidic mixer instrument, the NanoAssemblr® Ignite™ for testing. Briefly, 350 μL of mRNA was diluted using 100 mM sodium acetate buffer to the required concentration of 0.2 to 0.3 mg/mL. A lipid mix solution of 12.5 or 25 mM was typically used. LNP were then prepared by running both fluids, namely, nucleic acids in aqueous solvent and Lipid Mix in ethanol at a flow ratio of 3:1 and at a total flow rate of 12 ml/min in the microfluidic mixer. Following mixing in the microfluidic device, the post cartridge lipid nucleic acid particle sample was diluted into RNAse free tubes containing three to 40 volumes of PBS, pH 7.4. Ethanol was finally removed through either dialysis in PBS, pH 7, or using Amicon™ centrifugal filters (Millipore, USA) at 3000 RPM, or using TFF systems. Once the required concentration was achieved, the lipid nucleic acid particles were filter sterilized using 0.2 μm filters in aseptic conditions. Final encapsulation efficiency was measured by the Ribogreen® assay. Quant-iT™ RiboGreen® RNA Reagent and Kit (Invitrogen) following manufacturer directions. Self-amplifying mRNA plasmid NAT preparation is described below. Observed particle attributes were generally sized from 50-200 nm for mRNA, depending on lipid composition.

In Table 1, above, are listed the lipid compositions of the marketed LNP formulations ONPATTRO™, SPIKEVAX™ and COMIRNATY™. In Table 2 are listed a vaccine LNP formulation “Vaccmixb-PNI 516”, and the formulations according to the invention identified with the prefix starting from V46. These new vaccine formulations have a dramatically different ionizable lipid to phospholipid ratio than the compositions of Table 1 supra, and offer surprising advantage in terms of safety, since ionizable lipids can be irritating and PEG-lipids have been known to cause allergic reactions.

TABLE 2
Lipid Mixes for LNP

| | | Ratio Ionizable |
|---|---|---|
| Lipid Mix | Composition | lipid to DSPC |
| V22-PNI 516 | PNI 516: 40 mol % | 2 |
| | DSPC: 20 mol % | |
| | Cholesterol: 37.5 mol % | |
| | BRIJ ™ S10: 2.5 mol % | |
| Vaccmixb | PNI 516: 47.5 mol % | 3.80 |
| PNI 516 | DSPC: 12.5 mol % | |
| | Cholesterol: 38.5 mol % | |
| | PEG2000-DMG: 1.5 mol % | |
| V46-PNI 516 | PNI 516: 28.7 mol % | 0.58 |
| | DSPC: 49.8 mol % | |
| | Cholesterol: 20 mol % | |
| | PEG2000-DMG: 1.5 mol % | |
| V47-PNI 516 | PNI 516: 29.1 mol % | 0.59 |
| | DSPC: 50.6 mol % | |
| | Chol: 20.3 mol % | |
| | “PEG-free” | |
| V46-PNI 127 | PNI 127: 28.7 mol % | 0.58 |
| | DSPC: 49.8 mol % | |
| | Chol: 20 mol % | |
| | PEG2000-DMG 1.5 mol % | |
| V46-MC3 | MC3: 28.7 mol % | 0.58 |
| | DSPC: 49.8 mol % | |
| | Chol: 20 mol % | |
| | DMG-PEG: 1.5 mol % | |
| V46-PNI 550 | PNI 550: 28.7 mol % | 0.58 |
| | DSPC: 49.8 mol % | |
| | Chol: 20 mol % | |
| | PEG2000-DMG: 1.5 mol % | |
| V51-PNI 516 | PNI 516: 10 mol % | 0.54 |
| | DSPC: 18.5 mol % | |
| | Cholesterol: 70 mol % | |
| | DMG-PEG 1.5 mol % | |
| V51-PNI 127 | PN 127: 10 mol % | 0.54 |
| | DSPC: 18.5 mol % | |
| | Cholesterol: 70 mol % | |
| | DMG-PEG: 1.5 mol % | |
| V51-PNI 550 | PNI 550: 10 mol % | 0.54 |
| | DSPC: 18.5 mol % | |
| | Cholesterol: 70 mol % | |
| | DMG-PEG: 1.5 mol % | |
| V52-PNI 516 | PNI 516: 25.5 mol % | 0.47 |
| | DSPC: 54 mol % | |
| | Chol: 19 mol % | |
| | PEG2000-DMG: 1.5 mol % | |
| V53-PNI 516 | PNI 516: 25.6 mol % | 0.44 |
| | DSPC: 58.5 mol % | |
| | CHOL: 14.4 mol % | |
| | PEG2000_DMG 1.5 mol % | |
| V54-PNI 516 | PNI 516: 25 mol % | 0.50 |
| | DSPC: 49.6 mol % | |
| | CHOL: 23.9 mol % | |
| | PEG2000-DMG1.5 mol % | |
| V47-PNI 516 | PNI 516: 29.1 mol % | 0.58 |
| | DSPC: 50.6 mol % | |
| | CHOL: 20.3 mol % | |
| | PEG-free | |
| V52-PNI 516 | PNI 516: 10 mol % | 0.55 |
| | DSPC: 18.5 mol % | |
| | CHOL: 70 mol % | |
| | PEG2000-DMG: 1.5 mol % | |
| V53-PNI 516 | PNI-516: 10 mol % | 0.55 |
| | DSPC: 18 mol % | |
| | CHOL: 71.1 mol % | |
| | PEG-free | |
| V48-PNI 516 | PNI 516: 10 mol % | 0.54 |
| | DSPC: 18.5 mol % | |
| | Cholesterol: 70 mol % | |
| | PEG 2000 DMG: 1.5 mol % | |
| V88-PNI 516 | PNI 516: 10 mol % | 0.13 |
| | DSPC: 75 mol % | |
| | Cholesterol: 13.5 mol % | |
| | PEG 2000 DMG: 1.5 mol % | |
| V89-PNI 516 | PNI 516: 10 mol % | 0.29 |
| | DSPC: 34.9 mol % | |
| | Cholesterol: 53.6 mol % | |
| | PEG 2000 DMG: 1.5 mol % | |
| V92-PNI 721 | PNI 721: 10.5 mol % | 0.14 |
| | DSPC 75.5 mol % | |
| | Cholesteryl | |
| | hemisuccinate: 14.0 mol % | |

For Tables 3 and 4, all formulations were made with saRNA expressing Covid Spike protein. Both PNI 516 and MC3 (ionizable lipids) were formulated at a N/P of 8, with the RNA concentration for each formulation was kept constant at 84 ug/mL.

TABLE 3
Composition, Size, PDI, EE % and RNA Concentration

| | IL/PL | Run | | PNI516 | DSPC | Chol | Size | | EE |
|---|---|---|---|---|---|---|---|---|---|
| Lipid mix | ratio | Name | MC3% | mol % | mol % | mol % | (nm) | PDI | (%) |
| Vaccmixb (MC3) | 3.85 | PD-73-A | 50.0 | 0 | 10.0 | 38.5 | 53 | 0.11 | 96.3 |
| V46 (MC3) | 0.57 | PD-73-B | 28.7 | 0 | 49.8 | 20.0 | 69 | 0.14 | 53.9 |
| Vaccmixb-PNI | 3.85 | PD-73-C | 0 | 47.5 | 12.5 | 38.5 | 57 | 0.13 | 98.7 |
| 516 | | | | | | | | | |
| V46-PNI 516 | 0.57 | PD-73-D | 0 | 28.7 | 49.8 | 20.0 | 71 | 0.14 | 81.0 |
| V64-PNI516 | 0.74 | PD-73-E | 0 | 42.0 | 56.5 | 0.0 | 65 | 0.14 | 89.4 |
| V47-PNI516 | 0.57 | PD-73-F | 0 | 29.1 | 50.6 | 20.3 | 75 | 0.15 | 87.4 |
| V54 PNI 516 | 0.84 | PD-73-G | 0 | 26.8 | 31.9 | 39.8 | 70 | 0.17 | 96.8 |

TABLE 4
Size and PDI for MC3 v. PNI 516

| | | Size (nm) | | | | PDI | | | |
|---|---|---|---|---|---|---|---|---|---|
| | | | | | After | | | | After |
| | Run | 3x | Post | | Freeze | 3x | Post | | Freeze |
| Description | Name | diluted | Dialysis | Filtered | Thaw | diluted | Dialysis | Filtered | Thaw |
| Vaccixb-MC3 | PD- | 53 | 63 | 67 | 78 | 0.11 | 0.12 | 0.10 | 0.22 |
| | 073-A | | | | | | | | |
| V47-MC3 | PD- | 69 | 74 | 72 | 76 | 0.14 | 0.11 | 0.10 | 0.11 |
| | 073-B | | | | | | | | |
| VACCMIX-B- | PD- | 57 | 58 | 60 | 83 | 0.13 | 0.13 | 0.13 | 0.29 |
| PNI-516 | 073-C | | | | | | | | |
| V46-PNI516 | PD- | 71 | 71 | 74 | 81 | 0.14 | 0.14 | 0.16 | 0.16 |
| LM | 073-D | | | | | | | | |
| V47-PNI516 | PD- | 75 | 85 | 82 | 98 | 0.15 | 0.09 | 0.07 | 0.20 |
| | 073-F | | | | | | | | |

### Example 3

### Lipid Nucleic Acid Particle or “LNP” Characterization and Encapsulation

After the lipid particles were made as described above, the LNP particle size (hydrodynamic diameter of the particles) was determined by Dynamic Light Scattering (DLS) using a ZetaSizer™ Nano ZS™ (Malvern Instruments, UK). He/Ne laser of 633 nm wavelength was used as the light source. Data were measured from the scattered intensity data conducted in backscattering detection mode (measurement angle=173). Measurements were an average of 10 runs of two cycles each per sample. Z-Average size was reported as the particle size, and is defined as the harmonic intensity averaged particle diameter. Encapsulation efficiency was measured by a modified Ribogreen™ assay (Quanti-iT RiboGreen™ RNA assay kit, Fisher). There was good encapsulation in all the formulations (see Table 2 for composition), with polydispersity (PDI) under 0.19.

Post encapsulation steps were tangential flow filtration (TFF) concentration for about 40 minutes, and diafiltration. Effects on the LNP characteristics by these steps were minimal. Results not shown. LNP characteristics for the lipid mixes and LNP of the invention as well as controls are described in Tables 5 and 6.

TABLE 5
Effects on LNP (LNP formulated with SARS Cov2 Spike
saRNA and PNI 516 ionizable lipid) N/P ratio 8

| | | Ionizable | | | |
|---|---|---|---|---|---|
| | (PNI | lipid/ | | | |
| | 516:DSPC:CHOL:PEG- | DSPC | Size | | % |
| Lipid Mix | DMG2000) mol % | ratio | (nm) | PDI | EE |
| Vaccmixb- | 48.2:2.7:39.1:0 | 3.8 | 192 | 0.26 | 93 |
| PNI516 | | | | | |
| (Peg Free) | | | | | |
| V46-PNI 516 | 28.7:49.8:20:1.5 | 0.58 | 73 | 0.14 | 91 |
| V47-PNI 516 | 29.1:50.6:20.3:0 | 0.58 | 106 | 0.16 | 90 |
| V48-PNI 516 | 10:18.5:70:1.5 | 0.55 | 123 | 0.23 | 74 |
| V53-PNI 516 | 10:18:71.1:0 | 0.55 | 707 | 0.26 | 80 |

TABLE 6
Ratio of Phospholipid Effect on Encapsulation (LNP formulated
with SARS Cov2 saRNA and PNI 516 ionizable lipid).

| | | Ionizable | | | | |
|---|---|---|---|---|---|---|
| | | lipid/ | | | | |
| | Formulation | phospholipid | EC-50 | Size | | |
| Lipid Mix | (IL:DSPC:CHOL:DMG-PEG) | ratio | (pg/mL) | (nm) | PDI | % EE |
| V49-PNI 516 | 25.5:54:19:1.5 | 0.47 | 1.6 | 78 | 0.12 | 92 |
| V50-PNI 516 | 25.6:58.5:14.4:1.5 | 0.44 | 3.2 | 80 | 0.10 | 91 |
| V51-PNI 516 | 25:49.6:23.9:1.5 | 0.50 | 3.3 | 78 | 0.11 | 93 |
| V46-PNI 516 | 28.7:49.8:20:1.5 | 0.58 | 7.1 | 80 | 0.13 | 93 |
| Vaccmixb-PNI | 47.5:12.5:38.5:1.5 | 3.8 | 51.8 | 80 | 0.11 | 98 |
| 516 | | | | | | |

Note:
N/P ratio = 8

### Example 4

### High Throughput LNP Potency Assay in BHK Cells.

BHK 570 cells were purchased from ATCC, cultured as usual, and were transfected in a 96-well plate with saRNA A5 loaded LNP at 1000 to 0.49 ng/mL. LNP comprised of V46-PNI 516 showed a 12.6 fold improvement in EC₅₀ as compared to Vaccmixb-PNI 516. The V46 lipid mix showed an unexpected twelve times improvement in EC₅₀.

SARsCOV-2 protein expression was measured following BHK 570 cell transfection with various PNI 516 LNP formulations containing the nCoV PNI A5 saRNA as disclosed in PCT Pub. WO23057979 by Abraham et al.

TABLE 7
Comparison of EC50 for Two Lipid Mixes

| | Vaccmixb-PNI | V46-PNI |
|---|---|---|
| | 516 saRNA | 516 saRNA |
| EC₅₀ pg/mL | 25.2 | 2.0 |
| Fold improvements | 1 | 12.6 |
| over Vaccmixb-PNI516 | | |

A visual demonstration of SARsCOV-2 protein expression following transfection in BHK 570 cells can be made wherein green fluorescence represents the SARsCOV-2 protein stained with a fluorescent labelled anti SARsCOV-2 spike protein antibody (FAB105403G) following BHK 570 cells transfection at 31.25 ng/RNA well with Vaccmixb PNI 516 or V46-PNI 516. BHK 570 cells were plated in a 96-well plate (20 k cells/well) for 48 hours before transfection. Following transfection, the cells were incubated for a further 24 hours before being fixed with 4% PFA for 20 minutes. Cells were then permeabilized in 0.1% Triton X-100 and stained with a 1:50 dilution of FAB105403G antibody. Cells were imaged on a Cytation 7™ cell imaging multimode reader (BioTek, Agilent, Santa Clara, CA).

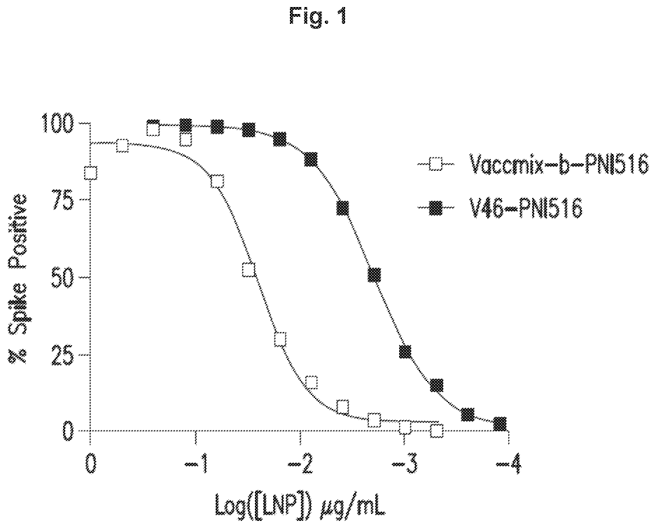

SARsCOV-2 protein expression dose response following Vaccmixb PNI 516 and V46-PNI 516 transfection in BHK 570 cells is graphically illustrated in FIG. 1. A dose response study was performed by testing a decreasing amount of RNA from 1000 to 0.49 ng/mL. BHK 570 cells were plated in a 96-well plate (20 k cells/well) for 48 hours before transfection. Following transfection, the cells were incubated for a further 24 hours before being fixed with 4% PFA for 20 minutes. Cells were then permeabilized in 0.1% Triton X-100 and stained with a 1:50 dilution of FAB105403G antibody. Cells were imaged on the Cytation 7™ (BioTek, Agilent, Santa Clara, CA) multimode reader.

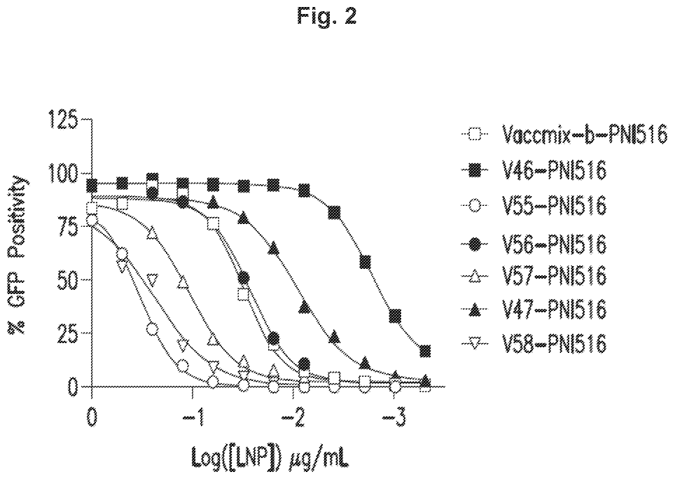

SARsCOV-2 protein expression was measured following BHK 570 cell transfection with various PNI516 LNP formulations containing the nCoV PNI A5 saRNA. A dose response study was performed by testing a decreasing amount of RNA from 1000 to 0.49 ng/mL. BHK 570 cells were plated in a 96-well plate (20 k cells/well) for 48 hours before transfection. Following transfection, the cells were incubated for a further 24 hours before being fixed with 4% PFA for 20 minutes. Cells were then permeabilized in 0.1% Triton X-100 and stained with a 1:50 dilution of FAB105403G antibody. Cells were imaged on the Cytation 7™. Results of the study are illustrated in FIG. 2.

For mRNA testing, a CLEANCAP™ EGFP mRNA (Trilink Biotechnologies, Cat. No. L-7601) was used, in a similar testing protocol as above. There were a variety of EC₅₀ readouts shown in the final column of Table 8 (N/P of 8) and Table 9 (N/P of 12), with lower levels indicating a greater therapeutic effect. This finding confirms a precise interplay among the ionizable lipid chemistry as well as the optimized ratios of components. “IL” is the ionizable lipid used. “NP” in the EC₅₀ column means the reading was too high to be meaningfully measured (negative result).

TABLE 8
BHK Cell EC50 ng/L testing for different ionizable
Lipids at a N/P Ratio of 8, mRNA

| | IL mol %/ | | | | | | |
|---|---|---|---|---|---|---|---|
| | DSPC mol %/ | | | | | | EC50 |
| | Chol mol %/ | IL/ | | Size | | | in BHK |
| Formulation | PEG DMG Mol % | DSPC | IL | nm | PDI | % EE | cells |
| V88 | 10:75:13.5:1.5 | 0.13 | PNI 127 | 153 | 0.336 | 65 | NP |
| | | | PNI 516 | 179 | 0.490 | 91 | NP |
| | | | PNI 550 | 188 | 0.472 | 90 | NP |
| | | | PNI 721 | 178 | 0.473 | 88 | 66 |
| | | | PNI 722 | 202 | 0.408 | 86 | 69 |
| | | | PNI 728 | 175 | 0.380 | 90 | 98 |
| V48 | 10:18.5:70:1.5 | 0.54 | PNI 127 | 117 | 0.335 | 61 | NP |
| | | | PNI 516 | 144 | 0.373 | 99 | NP |
| | | | PNI 550 | 119 | 0.185 | 97 | 503 |
| | | | PNI 560 | 130 | 0.242 | 100 | NP |
| | | | PNI 726 | 131 | 0.187 | 98 | 31 |
| | | | PNI 659 | 142 | 0.358 | 95 | 88 |
| | | | PNI 721 | 161 | 0.164 | 95 | 65 |
| | | | PNI 722 | 118 | 0.342 | 99 | 43 |
| | | | PNI 728 | 184 | 0.190 | 96 | 35 |
| | | | PNI 730 | 134 | 0.244 | 97 | NP |
| V46 | 28.7:49.8:20:1.5 | 0.58 | PNI 127 | 312 | 0.305 | 45 | NP |
| | | | PNI 516 | 184 | N/A | 89 | 301 |
| | | | PNI 550 | 100 | 0.251 | 96 | 72 |
| | | | PNI 659 | 122 | 0.268 | 90 | 61 |
| | | | PNI 721 | 132 | 0.258 | 77 | 62 |
| | | | PNI 723 | 123 | 0.347 | 90 | 86 |
| | | | PNI 728 | 140 | 0.360 | 87 | 42 |
| V47 | 29.1:50.6:20.3:0 | 0.58 | PNI 127 | 52 | 0.369 | 61 | NP |
| | | | PNI 516 | 152 | 0.103 | 84 | NP |
| | | | PNI 550 | 132 | 0.462 | 94 | 151 |
| | | | PNI 721 | 165 | 0.286 | 68 | 69 |
| V89 | 10:34.9:53.6:1.5 | 0.29 | PNI 127 | 95 | 0.323 | 76 | N/A |
| | | | PNI 516 | 138 | 0.314 | 97 | NP |
| | | | PNI 560 | 132 | 0.297 | 97 | 53 |

In Table 8, there is a large variation in efficacy for the inventive formulation depending on the ionizable lipid used. EC₅₀ is a measure of the efficacy of the LNP to transfect cells and LNP contents to express the GOI. The lower the value, the more effective the formulation.

TABLE 9
BHK Cell EC50 ng/L testing for different Ionizable
Lipids at a N/P Ratio of 12, mRNA

| | IL mol %/ | | | | | |
|---|---|---|---|---|---|---|
| | DSPC mol %/ | | | | | EC50 |
| | Chol mol %/ | IL/ | | Size | | in BHK |
| Formulation | PEG DMG Mol % | DSPC | IL | nm | % EE | cells |
| V88 | 10:75:13.5:1.5 | 0.13 | PNI 127 | 206 | 80 | 3208 |
| | | | PNI 516 | 97 | 96 | 4 |
| | | | PNI 580 | 233 | 81 | 282 |
| | | | PNI 560 | 192 | 84 | 599 |
| | | | PNI 726 | 165 | 89 | NP |
| | | | PNI 659 | 193 | 74 | 2092 |
| V48 | 10:18.5:70:1.5 | 0.54 | PNI 127 | 254 | 57 | 124 |
| | | | PNI 516 | 119 | 98 | 63 |
| | | | PNI 580 | 165 | 97 | 8227 |
| | | | PNI 560 | 135 | 96 | NP |
| | | | PNI 726 | 132 | 99 | 8 |
| | | | PNI 659 | 90 | 65 | 168 |
| V46 | 28.7:49.8:20:1.5 | 0.58 | PNI 127 | 181 | 45 | 190 |
| | | | PNI 516 | 92 | 93 | 220 |
| | | | PNI 580 | 2198 | 4 | 276 |
| | | | PNI 560 | 159 | 91 | 577 |
| | | | PNI 726 | 1024 | 21 | 267 |
| V47 | 29.1:50.6:20.3:0 | 0.58 | PNI 127 | 130 | 53 | 156 |
| | | | PNI 516 | 130 | 86 | 108 |
| | | | PNI 659 | 190 | 78 | 69 |
| V89 | 10:34.9:53.6:1.5 | .29 | PNI 127 | 139 | 88 | 855700 |
| | | | PNI 516 | 100 | 97 | Unstable |
| | | | PNI 560 | 131 | 95 | 58 |

TABLE 10
Combined Best Ionizable lipids for each
composition at N/P 8 and N/P12.

| Composition (ratios) | N/P 8 | N/P 12 |
|---|---|---|
| V46 | PNI 550, PNI 659, PNI 721, | None |
| | PNI 723, PNI 728 | |
| V47 | PNI 721 | PNI 516, PNI 659 |
| V48 | PNI 127, PNI 516 PNI 550, | PNI 516, PNI 726 |
| | PNI 560, PNI 659, PNI 721, | |
| | PNI 722, PNI 726, PNI 728, | |
| V88 | PNI 721, PNI 722, PNI 728 | PNI 516 |
| V89 | PNI 560 | PNI 560 |

### Example 5

### In Vivo Study

SARsCOV-2 spike protein specific IgG expression in mouse serum was measured following intramuscular LNP injection to BALB/c mice at 1 ug/mouse. Enzyme-linked immunosorbent assay (ELISA) was performed according to the established methods for measuring SARS-CoV-2 spike protein-specific IgG antibodies in serum samples collected from animals on day 21 and 42 post immunization.

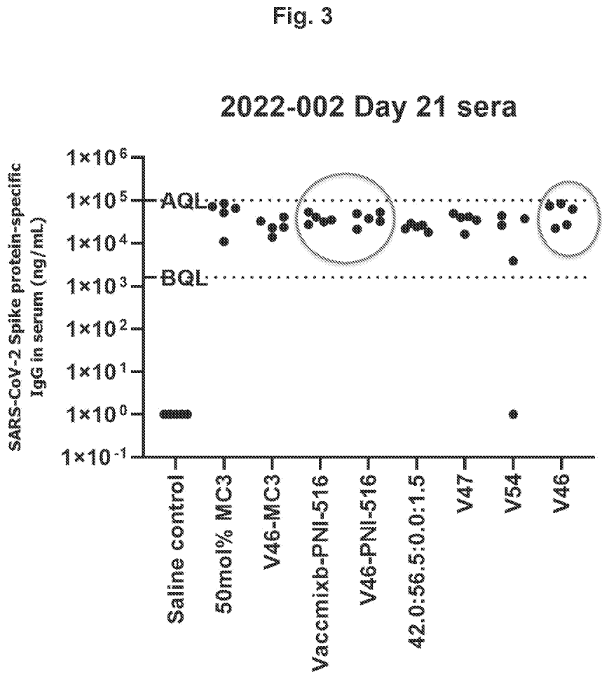

Reagents for the ELISA: DPBS 1×Sterile (pH7.4) without Ca2+ and Mg2+, Corning; assay diluent B (5×); Purified anti-SARS-CoV-2 S Protein S1 Antibody Rat; ELISA wash buffer (20×); HRP Goat anti-ratlgG (minimal x-reactivity) Antibody; and TMB substrate solution, (all from BioLegend, San Diego, CA); Isopropyl Alcohol (IPA), 70% and Water (Nuclease Free) (from VWR); Nunc MaxiSorp™ flat-bottom 96-well plate and Stop solution (Thermo Fisher); and SARS-CoV-2 (2019-nCoV) spike S1+S2 ECD-His recombinant protein, (Sino Biologicals). FIG. 3 illustrates the IgG levels for the different groups on day 21. On day 42, results were beneficially about ten-fold higher (not shown), which is a good result.

### Example 6

### Three Month Stability Study

This study was performed for V46 with PNI 516 as the ionizable lipid. Ten aliquots of frozen LNP (50 ug/mL saRNA) in pH 7.4 buffer were thawed to room temperature and then stored in the dark for 1 h, 2 h, 4 h, 24 h, 2 days, 3 days, 4 days and 7 days, one month and three months. At each time point, the LNP particle size was measured by dynamic light scattering and the percent saRNA encapsulation was determined using a Quant-it™ RiboGreen RNA Assay Kit™ (Thermo Fisher). Results are shown in Table 11.

TABLE 11
Stability at Room Temperature of V46-PNI 516 LNP

| Time at RT | Size (nm) | PDI | % Encap. |
|---|---|---|---|
| T = 0 | 80 | 0.14 | 92 |

| | | | | |
|---|---|---|---|---|
| 24 | Hours | 83 | 0.13 | 93 |
| 48 | Hours | 84 | 0.14 | 93 |
| 72 | Hours | 83 | 0.14 | 92 |
| 1 | Week | 84 | 0.14 | 93 |
| 1 | Month | 84 | 0.12 | 95 |
| 3 | Months | 84 | 0.14 | 92 |

### Example 7

### Cryo-Transmission Electron Microscopy (CryoTEM) Structure of LNP

LNPs comprising PNI 516, DSPC, cholesterol and PEG-DMG encapsulating saRNA were cryopreserved, sectioned, and prepared for Cryo-TEM examination. Cryo-TEM of LNP comprising the standard IL:DSPC ratio of 3.8 resulted in mostly dense unilamellar vesicles with some multi-compartmental vesicles, while LNP comprising IL:DSPC with DMG-PEG ratio of 0.58 showed multilayers vesicles with higher numbers of multi-compartments vesicles.

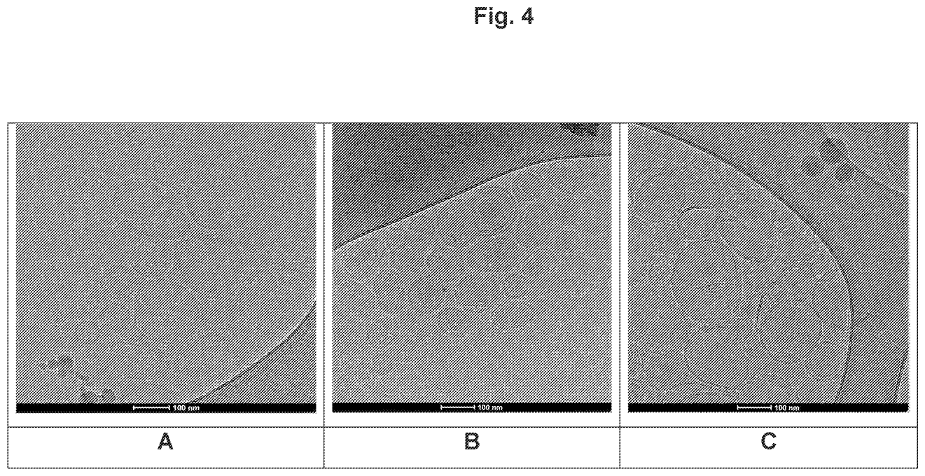

The peg free LNP with the IL:DSPC ratio of 0.58 also showed more multi-lamellar vesicles but particles are closer together as compared to the pegylated version of this composition. FIG. 4 shows exemplary images of A, Vaccmixb-PNI 516; B, V46-PNI 516; and C, V47-PNI 516.

### Example 8

### LNP Stability and Activity Following Lyophilization

In this experiment, 2 mL glass vials were filled with 500 uL of LNP formulations described below, each with PNI 516 as ionizable lipid, then the LNP samples were equilibrated to a temperature of 5 deg. C and a pressure of 750 Torr. After that, the temperature was reduced from 5 deg. C to −50 deg. C at 400-500 Torr. Samples were kept at −50 deg. C for 3 hours. Subsequently, pressure was dropped to 0.062 Torr at −50 deg. C to initiate primary drying for 120 minutes, then the shelf temperature was increased to −40 deg. C at a pressure of 0.062 Torr and kept at those conditions for 12 hours (720 min). Lastly, a secondary drying was performed at 10 deg. C, 0.062 Torr for 180 minutes.

After the lyophilization, samples were sealed and stored at 4 deg. C. The next day, samples were reconstituted with 500 μL of molecular biology grade water. The rehydrated LNP were then analyzed for particle size, SARNA encapsulation and biological activity as described above.

The formulation EC₅₀ was determined using a dose response in vitro by testing a decreasing amount of RNA from 1000 to 0.49 ng/mL. BHK 570 cells were plated in a 96-well plate (20 k cells/well) for 48 hours before transfection. Following transfection, the cells were incubated for a further 24 hours before being fixed with 4% PFA for 20 minutes. Cells were then permeabilized in 0.1% Triton X-100 (Sigma Aldrich) and stained with a 1:50 dilution of FAB105403G antibody (Bio-Techne, Toronto, Ontario). Cells were imaged on the Cytation 7™ (Agilent, Santa Clara, CA) reader.

The lyophilized version of V46-PNI 516 LNP and V47-PNI 516 did not show significant changes in particle size after rehydration, in contrast to Vaccmixb PNI516 which significantly grew in size. In addition, the Vaccmixb PNI516 showed a 7-fold decrease in activity as compared to V46-PNI 516 LNP and V47-PNI 516 in BHK 570 cell transfection.

TABLE 12
Size, PDI, Encapsulation Percent, and EC50 Before and After Lyophilization

| | | | | | | | | | | Fold |
|---|---|---|---|---|---|---|---|---|---|---|
| Formulation | | Size (nm) | | PDI | | % Encap. | | EC₅₀ (pg/mL) | | change in |
| | IL/PL | Pre | Post | Pre | Post | Pre | Post | Pre | Post | activity |
| Code | ratio | Lyo | Lyo | Lyo | Lyo | Lyo | Lyo | Lyo | Lyo | after lyo |
| Vaccmixb | 3.85 | 80 | 166 | 0.18 | 0.06 | 98 | 92 | 99.5 | 682.4 | −6.9 |
| PNI516 | | | | | | | | | | |
| V46-PNI | 0.57 | 81 | 97 | 0.12 | 0.1 | 92 | 92 | 73.7 | 19.5 | 0.3 |
| 516 | | | | | | | | | | |
| V47-PNI | 0.57 | 123 | 137 | 0.18 | 0.21 | 89 | 90 | 316.5 | 372.9 | 1.2 |
| 516 | | | | | | | | | | |

Ionizable lipid combinations in the high phospholipid compositions according to the invention were made as described above. The LNP particle size (hydrodynamic diameter of the particles) and encapsulation efficiency were measured by a modified Ribogreen™ assay was performed as described above. There was good encapsulation size and PDI in all the formulations (see Table 13 for composition), with polydispersity (PDI) under 0.25 size<117 nm. The EC₅₀ was determined as described above in BHK 570 cells using an in vitro dose response by testing a decreasing amount of RNA from 1 to 0.00049 μg/well.

Table 13 illustrates the composition and ratios of various mixtures of ionizable lipids, and the effect of LNP made therefrom on GFP saRNA EC50 and the difference between EC 50 results from Vaccmixb (control) EC50. V76 and V68, representing combinations of ionizable lipids, showed exceptionally good EC50 pg/ml) scores. V75, V69, V46, V74, V76 all representing ionizable lipid mixtures were also very good compared to controls.

TABLE 12
Compositions and EC50 Compared to “Benchmark”
Vaccmixb EC₅₀ (PNI GFP saRNA

| | | PNI | PNI | PNI | | | | EC |
|---|---|---|---|---|---|---|---|---|
| | Total | 127 | 550 | 516 | DSPC | Chol | EC₅₀ | difference |
| Lipid | IL/PL | (mol | (mol | (mol | (mol | (mol | (pg/ | from |
| Mix | ratio | %) | %) | %) | %) | %) | mL) | Vaccmixb |
| V76-PNI | 0.58 | 0 | 21.6 | 7.2 | 49.8 | 20 | 3.0 | 22.1 |
| 550/PNI | | | | | | | | |
| 516 | | | | | | | | |
| V68-PNI | 0.58 | 14.4 | 0 | 14.4 | 49.8 | 20 | 4.0 | 18 |
| 127/PNI | | | | | | | | |
| 516 | | | | | | | | |
| V75-PNI | 0.58 | 0 | 7.2 | 21.5 | 49.8 | 20 | 5.0 | 13.8 |
| 550/PNI | | | | | | | | |
| 516 | | | | | | | | |
| V69-PNI | 0.58 | 7.2 | 0 | 21.5 | 49.8 | 20 | 5.0 | 13.5 |
| 127/PNI | | | | | | | | |
| 516 | | | | | | | | |
| V46-PNI | 0.58 | 0 | 0 | 28.7 | 49.8 | 20 | 5.0 | 13.5 |
| 516 | | | | | | | | |
| V74-PNI | 0.58 | 0 | 14.4 | 14.4 | 49.8 | 20 | 6.0 | 11.5 |
| 550/PNI | | | | | | | | |
| 516 | | | | | | | | |
| V67-PNI | 3.81 | 35.8 | 0 | 11.8 | 12.5 | 38.5 | 6.0 | 11.5 |
| 127/PNI | | | | | | | | |
| 516 | | | | | | | | |
| V46-PNI | 0.58 | 28.7 | 0 | 0 | 49.8 | 20 | 11.0 | 6.7 |
| 127 | | | | | | | | |
| V70-PNI | 0.58 | 21.6 | 0 | 7.2 | 49.8 | 20 | 11.0 | 6.4 |
| 127/PNI | | | | | | | | |
| 516 | | | | | | | | |
| V46-PNI | 0.58 | 0 | 28.7 | 0 | 49.8 | 20 | 14.0 | 5 |
| 550 | | | | | | | | |
| Vaccmixb- | 3.80 | 0 | 47.5 | 0 | 12.5 | 38.5 | 21.0 | 3.4 |
| PNI 550 | | | | | | | | |
| V66-PNI | 3.81 | 11.8 | 0 | 35.8 | 12.5 | 38.5 | 26.0 | 2.7 |
| 127/PNI | | | | | | | | |
| 516 | | | | | | | | |
| Vaccmixb- | 3.80 | 47.5 | 0 | 0 | 12.5 | 38.5 | 26.0 | 2.7 |
| PNI 127 | | | | | | | | |
| V65-PNI | 3.81 | 23.8 | 0 | 23.8 | 12.5 | 38.5 | 29.0 | 2.5 |
| 127/PNI | | | | | | | | |
| 516 | | | | | | | | |
| V73-PNI | 3.81 | 0 | 35.8 | 11.8 | 12.5 | 38.5 | 32.0 | 2.2 |
| 550/PNI | | | | | | | | |
| 516 | | | | | | | | |
| V71-PNI | 3.81 | 0 | 23.8 | 23.8 | 12.5 | 38.5 | 41.0 | 1.7 |
| 550/PNI | | | | | | | | |
| 516 | | | | | | | | |
| V72-PNI | 3.81 | 0 | 11.8 | 35.8 | 12.5 | 38.5 | 54.0 | 1.3 |
| 550/PNI | | | | | | | | |
| 516 | | | | | | | | |
| Vaccmixb- | 3.80 | 0 | 0 | 47.5 | 12.5 | 38.5 | 71.0 | 1 |
| PNI 516 | | | | | | | | |

The mixed ionizable lipid mixes were also tested for the physical characteristics of the LNP. Results are shown in Table 13.

TABLE 13
Physical characteristics for the new compositions
containing a mixture of ionizable lipid

| | | Size | | % |
|---|---|---|---|---|
| Code | Run # | (nm) | PDI | Encap. |
| Vaccmixb-PNI 516 | PD-80-1 | 61 | 0.16 | 98 |
| Vaccmixb-PNI 127 | PD-80-2 | 116 | 0.25 | 83 |
| V65-PNI 127/PNI 516 | PD-80-3 | 73 | 0.25 | 97 |
| V66-PNI 127/PNI 516 | PD-80-4 | 78 | 0.23 | 97 |
| V67-PNI 127/PNI5 16 | PD-80-5 | 117 | 0.25 | 85 |
| V46-PNI 516 | PD-80-6 | 73 | 0.15 | 96 |
| V46-PNI 127 | PD-80-7 | 83 | 0.13 | 95 |
| V68-PNI 127/PNI 516 | PD-80-8 | 79 | 0.14 | 95 |
| V69-PNI 127/PNI 516 | PD-80-9 | 76 | 0.13 | 95 |
| V70-PNI 127/PNI 516 | PD-80-10 | 83 | 0.13 | 94 |
| Vaccmixb-PNI 550 | PD-80-11 | 71 | 0.21 | 97 |
| V71-PNI 550/PNI 516 | PD-80-12 | 75 | 0.24 | 97 |
| V72-PNI 550/PNI 516 | PD-80-13 | 70 | 0.20 | 98 |
| V73-PNI 550/PNI 516 | PD-80-14 | 74 | 0.23 | 97 |
| V46-PNI 550 | PD-80-15 | 69 | 0.12 | 98 |
| V74-PNI 550/PNI 516 | PD-80-16 | 70 | 0.12 | 96 |
| V75-PNI 550/PNI 516 | PD-80-17 | 76 | 0.10 | 95 |
| V76-PNI 550/PNI 516 | PD-80-18 | 61 | 0.16 | 98 |

### Example 9

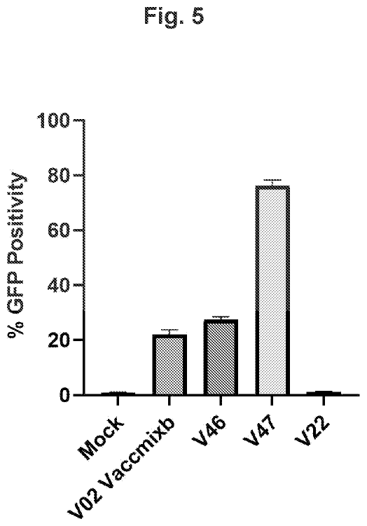

This Example demonstrates the in vitro transfection ability in Jurkat cells of saRNA LNPs made with different lipid mixes. Following transfection at 0.32 ug/mL of V02 Vaccmixb, V46-PNI516, V47-PNI516 and V22-PNI516 in Jurkat cells, % GFP positivity was evaluated using flow cytometry. The LNPs were tested by adding Jurkat cells (50 k/well) to GFP saRNA LNP to a final saRNA concentration of 3.2 ug/mL. Following a 24 hour incubation, cells were resuspended in a BSA stain buffer and analyzed using BECKMAN COULTER™ Cytoflex S flow cytometer. Results are shown in FIG. 5.

### Example 10

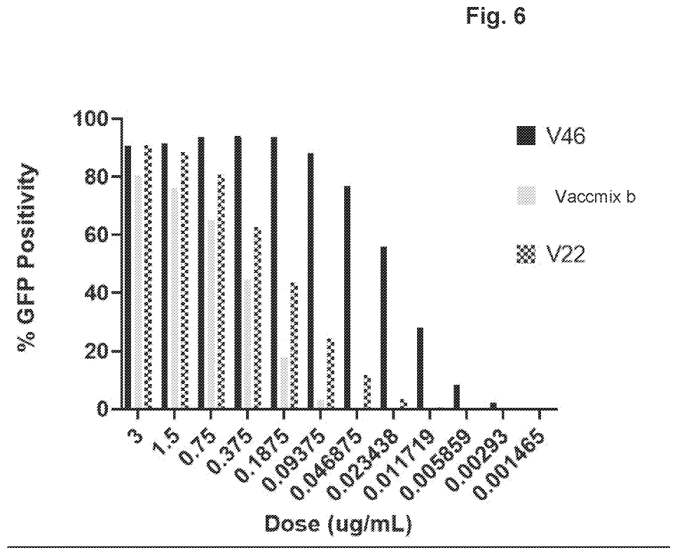

This Example demonstrates the in vitro transfection ability in Jurkat cells of mRNA LNPs made with different lipid mixes. Following transfection of V46-PNI516, V02-PNI516 and V22-PNI516 in Jurkat cells, % GFP positivity was evaluated in a dose response experiment. The dose response was tested by adding Jurkat cells (50 k/well) to a decreasing amount of GFP mRNA LNP from 3 to 0.001465 ug/well. Following a 24 hour incubation, cells were re-suspended in a BSA stain buffer and analyzed using Beckman Coulter Cytoflex S flow cytometer. Results are shown in FIG. 6.

### Example 11

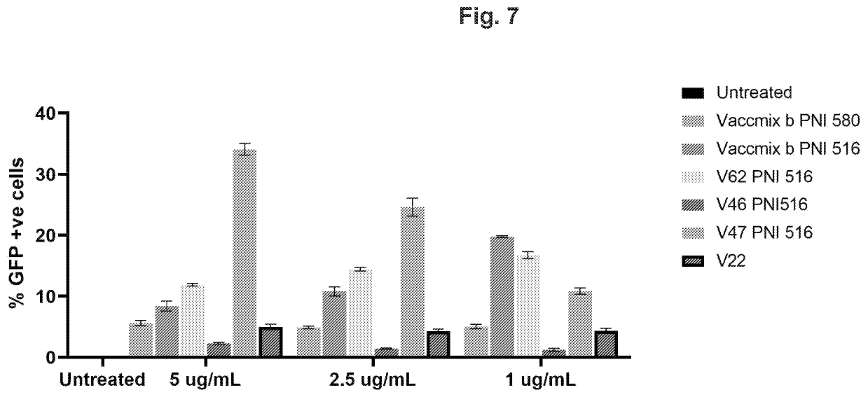

This Example demonstrates the transfection ability in T cells isolated from PBMC of EGFP saRNA LNPs made with different lipid mixes. Human PBMCs were activated for 3 days in ImmunoCult-XF T cell expansion medium supplemented with activators/recombinant human IL-2. After the activation, cells were diluted to a concentration of 0.25×106 cells/ml along with ApoE. LNP formulations (mentioned in FIG. 7) containing EGFP saRNA were supplemented to the cell culture media and the transfection was carried out for 24 hrs. CD4+ and CD8+ T cells were gated using specific antibodies and % GFP positivity in the T cells were evaluated using standard flow cytometry analysis. Dead cells were disregarded from the analysis using viability stain. Dead cells are susceptible to giving nonspecific staining with the fluorescent antibodies and are generally excluded by gating them out. Results are shown in FIG. 7.

While specific embodiments of the invention have been described and illustrated, such embodiments should be considered illustrative of the invention only and not as limiting the invention as construed in accordance with the accompanying claims.

### Example 12

This Example demonstrates the efficacy of the LNP of the present invention in an intravenous protein replacement model.

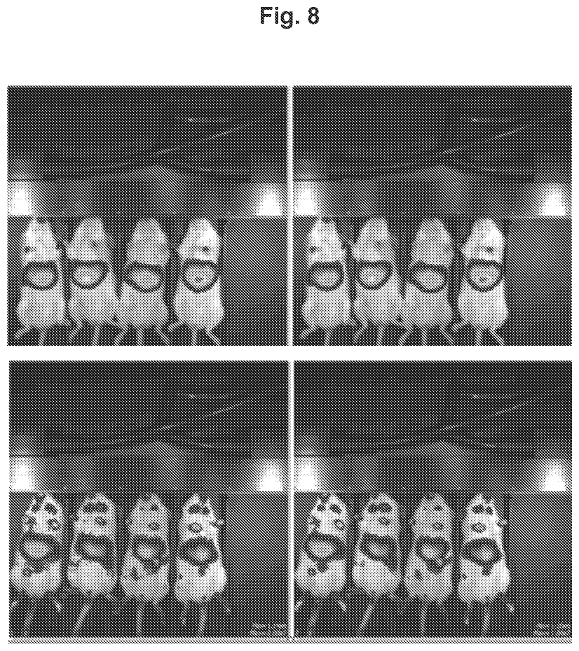

Two different LNPs according to embodiments of the invention were injected into mice intravenously at 0.1 mg/kg. The payload was Luciferase·mRNA (Trilink-CLEANCAP™ FLuc mRNA (5 moU), Cat #L-7202). The top row of photographs in FIG. 8 showing fluorescence at four hours post administration was achieved by intravenous administration of LNP comprising PNI 516 30%, DPSC 56%, cholesterol 12.5% and DMG-PEG 1.5%. The bottom row in FIG. 8, which is brighter, was achieved by LNP comprising PNI 580 30%, DSPC 56%, cholesterol 12.5%, and DMG-PEG-1.5%. This demonstrates the LNPs of the invention successfully express protein in vivo after intravenous administration. PNI 580 appears slightly more effective at enabling non-native protein expression than PNI 516 in this model.

1. A lipid formulation capable of forming a lipid-based nanoparticle suitable for vaccines comprising an ionizable lipid to phospholipid ratio of 0.1 Mol:Mol-1.3 Mol:Mol.

2. The lipid formulation of claim 1, further including nucleic acid payload.

3. The lipid formulation of claim 2, wherein the nucleic acid payload is a nucleic acid vaccine element.

4. The lipid formulation of any of claims 1-3, further comprising a stabilizer.

5. The lipid formulation of any of claims 1-4, further comprising a sterol.

6. The lipid formulation of any of claims 1-5, wherein the ionizable lipid to phospholipid lipid ratio is 0.4 Mol:Mol-0.70 Mol:Mol.

7. The lipid formulation of any of claims 1-6, wherein the ionizable lipid to phospholipid ratio is about 0.70.

8. The lipid formulation of any of claims 1-6, wherein the ionizable lipid to phospholipid ratio is about 0.69.

9. The lipid formulation of any of claims 1-6, wherein the ionizable lipid to phospholipid ratio is about 0.68.

10. The lipid formulation of any of claims 1-6, wherein the ionizable lipid to phospholipid ratio is about 0.67.

11. The lipid formulation of any of claims 1-6, wherein the ionizable lipid to phospholipid ratio is about 0.66.

12. The lipid formulation of any of claims 1-6, wherein the ionizable lipid to phospholipid ratio is about 0.65.

13. The lipid formulation of any of claims 1-6, wherein the ionizable lipid to phospholipid ratio is about 0.64.

14. The lipid formulation of any of claims 1-6, wherein the ionizable lipid to phospholipid ratio is about 0.63.

15. The lipid formulation of any of claims 1-6, wherein the ionizable lipid to phospholipid ratio is about 0.62.

16. The lipid formulation of any of claims 1-6, wherein the ionizable lipid to phospholipid ratio is about 0.61.

17. The lipid formulation of any of claims 1-6 wherein the ionizable lipid to phospholipid ratio is about 0.60.

18. The lipid formulation of any of claims 1-6 wherein the ionizable lipid to phospholipid ratio is about 0.59.

19. The lipid formulation of any of claims 1-6 wherein the ionizable lipid to phospholipid ratio is about 0.58.

20. The lipid formulation of any of claims 1-6 wherein the ionizable lipid to phospholipid ratio is about 0.57.

21. The lipid formulation of any of claims 1-6 wherein the ionizable lipid to phospholipid ratio is about 0.56.

22. The lipid formulation of any of claims 1-6 wherein the ionizable lipid to phospholipid ratio is about 0.55.

23. The lipid formulation of any of claims 1-6 wherein the ionizable lipid to phospholipid ratio is about 0.54.

24. The lipid formulation of any of claims 1-6 wherein the ionizable lipid to phospholipid ratio is about 0.53.

25. The lipid formulation of any of claims 1-6 wherein the ionizable lipid to phospholipid ratio is about 0.52.

26. The lipid formulation of any of claims 1-6 wherein the ionizable lipid to phospholipid ratio is about 0.51.

27. The lipid formulation of any of claims 1-6 wherein the ionizable lipid to phospholipid ratio is about 0.50.

28. The lipid formulation of any of claims 1-6 wherein the ionizable lipid to phospholipid ratio is about 0.49.

29. The lipid formulation of any of claims 1-6 wherein the ionizable lipid to phospholipid ratio is about 0.48.

30. The lipid formulation of any of claims 1-6 wherein the ionizable lipid to phospholipid ratio is about 0.47.

31. The lipid formulation of any of claims 1-6 wherein the ionizable lipid to phospholipid ratio is about 0.46.

32. The lipid formulation of any of claims 1-6 wherein the ionizable lipid to phospholipid ratio is about 0.45.

33. The lipid formulation of any of claims 1-6 wherein the ionizable lipid to phospholipid ratio is about 0.44.

34. The lipid formulation of any of claims 1-6 wherein the ionizable lipid to phospholipid ratio is about 0.43.

35. The lipid formulation of any of claims 1-6 wherein the ionizable lipid to phospholipid ratio is about 0.42.

36. The lipid formulation of any of claims 1-6 wherein the ionizable lipid to phospholipid ratio is about 0.41.

37. The lipid formulation of any of claims 1-6 wherein the ionizable lipid to phospholipid ratio is about 0.40.

38. The lipid formulation of any of claims 1-5 wherein the ionizable lipid to phospholipid ratio is about 0.30 to 0.39.

39. The lipid formulation of any of claims 1-5 wherein the ionizable lipid to phospholipid ratio is about 0.20 to 0.29.

40. The lipid formulation of any of claims 1-5 wherein the ionizable lipid to phospholipid ratio is about 0.10 to 0.19.

41. The lipid formulation of claim 1, wherein the ionizable lipid comprises 20-40 mol % of the total lipid.

42. The lipid formulation of any of claims 1-41, wherein the ionizable lipid comprises a mixture of ionizable lipids.

43. The lipid formulation of claim 1, wherein the phospholipid comprises from 40-60 mol %.

44. The lipid formulation of any of claims 5-43, wherein the sterol comprises from 15-25 mol %.

45. The lipid formulation of any of claims 5-44, wherein the sterol is cholesterol.

46. The lipid formulation of any of claims 5-44, wherein the sterol is cholesteryl hemisuccinate.

47. The lipid formulation of any of claims 4-46, wherein the stabilizer comprises from 0.0-2.5 mole %.

48. The lipid formulation of any of claims 4-47, wherein the stabilizer is a PEG lipid.

49. The lipid formulation of any of claims 1-48, wherein the phospholipid is DSPC.

50. The lipid formulation of any of claims 1-49, wherein the ionizable lipid is selected from the group consisting of PNI 127, PNI 516, PNI 550, PNI 560, PNI 580, PNI 659, PNI 721, PNI 722, PNI 726, PNI 728, and PNI 730.

51. The lipid formulation of claim 50, wherein the ionizable lipid is selected from the group consisting of PNI 516, PNI 550, and PNI 127.

52. A lipid formulation for encapsulating a nucleic acid payload in a vaccine nanoparticle, the formulation comprising an ionizable lipid, a sterol, and a phospholipid, wherein the phospholipid content is between 25-60% and the ionizable lipid to phospholipid molar ratio is from 0.33 to 1.2.

53. A vaccine comprising the lipid formulation of any of claims 1-52 wherein the nucleic acid vaccine element codes for an antigen selected from coronavirus spike protein and influenza hemagglutinin protein.

54. A vaccine comprising the lipid formulation of any of claims 1-53 wherein the nucleic acid vaccine element is derived from influenza virus.

55. A vaccine comprising the lipid formulation of any of claims 1-54 wherein the nucleic acid vaccine element is derived from coronavirus.
